SEQUENCE LISTING (24 sequences)

SEQ ID NO: 1 - protein, 4 aa, synthetic construct
  CHAIN 1..4: note = wherein, the side chain of amino acids are free or protected with a suitable protecting group
TFTS

SEQ ID NO: 2 - protein, 6 aa, synthetic construct
  CHAIN 1..6: note = wherein, the side chain of amino acids are free or protected with a suitable protecting group
  SITE 5: note = Aib; note = Aib
DYSIXL

SEQ ID NO: 3 - protein, 12 aa, synthetic construct
  CHAIN 1..12: note = wherein, the side chain of amino acids are free or protected with a suitable protecting group
  SITE 3: note = Lys* is protected using iVDde
AQKAFVQWLIAG

SEQ ID NO: 4 - skipped

SEQ ID NO: 5 - protein, 8 aa, synthetic construct
  CHAIN 1..8: note = wherein, the side chain of amino acids are free or protected with a suitable protecting group
  SITE 2: note = Aib
YXEGTFTS

SEQ ID NO: 6 - protein, 11 aa, synthetic construct
  CHAIN 1..11: note = wherein, the side chain of amino acids are free or protected with a suitable protecting group
  SITE 2: note = Aib
YXEGTFTSDYS

SEQ ID NO: 7 - protein, 10 aa, synthetic construct
  CHAIN 1..10: note = wherein, the side chain of amino acids are free or protected with a suitable protecting group
  SITE 2: note = Aib
IXLDKIAQKA

SEQ ID NO: 8 - protein, 14 aa, synthetic construct
  CHAIN 1..14: note = wherein, the side chain of amino acids are free or protected with a suitable protecting group
  SITE 5: note = Lys* is a side chain present In Tirzepatide at Lys (20 position)
KIAQKAFVQWLIAG

SEQ ID NO: 9 - skipped

SEQ ID NO: 10 - protein, 18 aa, synthetic construct
  CHAIN 1..18: note = wherein, the side chain of amino acids are free or protected with a suitable protecting group
  SITE 2: note = Aib
  SITE 9: note = Lys* is a side chain present In Tirzepatide at Lys (20 position)
IXLDKIAQKAFVQWLIAG

SEQ ID NO: 11 - protein, 37 aa, synthetic construct
  CHAIN 1..37: note = wherein, the side chain of amino acids are free or protected with a suitable protecting group
  SITE 18: note = Lys* is a side chain present In Tirzepatide at Lys (20 position)
  SITE 11: note = Aib
  SITE 1: note = NH2-Glu
  SITE 37: note = Ser-CONH2
EGTFTSDYSIXLDKIAQKAFVQWLIAGGPSSGAPPPS

SEQ ID NO: 12 - protein, 38 aa, synthetic construct
  CHAIN 1..38: note = wherein, the side chain of amino acids are free or protected with a suitable protecting group
  SITE 19: note = Lys* is a side chain present In Tirzepatide at Lys (20 position)
  SITE 1: note = Aib
  SITE 12: note = Aib
  SITE 1: note = NH2--Aib
  SITE 38: note = Ser-CONH2
XEGTFTSDYSIXLDKIAQKAFVQWLIAGGPSSGAPPPS

SEQ ID NO: 13 - protein, 39 aa, synthetic construct
  CHAIN 1..39: note = wherein, the side chain of amino acids are free or protected with a suitable protecting group
  SITE 20: note = Lys* is a side chain present In Tirzepatide at Lys (20 position)
  SITE 1: note = D-Tyrosine
  SITE 2: note = Aib
  SITE 1: note = NH2-D-Tyr
  SITE 39: note = Ser-CONH2
  SITE 13: note = Aib
YXEGTFTSDYSIXLDKIAQKAFVQWLIAGGPSSGAPPPS

SEQ ID NO: 14 - protein, 39 aa, synthetic construct; identical to SEQ ID NO: 13
  CHAIN 1..39: note = wherein, the side chain of amino acids are free or protected with a suitable protecting group
  SITE 20: note = Lys* is a side chain present In Tirzepatide at Lys (20 position)
  SITE 1: note = NH2-Tyr
  SITE 2: note = Aib
  SITE 39: note = Ser-CONH2
  SITE 8: note = D-Serine
  SITE 13: note = Aib

SEQ ID NO: 15 - protein, 39 aa, synthetic construct; identical to SEQ ID NO: 13
  CHAIN 1..39: note = wherein, the side chain of amino acids are free or protected with a suitable protecting group
  SITE 20: note = Lys* is a side chain present In Tirzepatide at Lys (20 position) with alpha-Glutamic acid
  SITE 1: note = NH2-Tyr
  SITE 2: note = Aib
  SITE 39: note = Ser-CONH2
  SITE 13: note = Aib

SEQ ID NO: 16 - protein, 39 aa, synthetic construct; identical to SEQ ID NO: 13
  CHAIN 1..39: note = wherein, the side chain of amino acids are free or protected with a suitable protecting group
  SITE 1: note = NH2-Tyr
  SITE 2: note = Aib
  SITE 39: note = Ser-CONH2
  SITE 13: note = Aib

SEQ ID NO: 17 - protein, 39 aa, synthetic construct; identical to SEQ ID NO: 13
  CHAIN 1..39: note = wherein, the side chain of amino acids are free or protected with a suitable protecting group
  SITE 20: note = Lys* is protected using IVDde
  SITE 1: note = NH2-Tyr
  SITE 2: note = Aib
  SITE 13: note = Aib
  SITE 39: note = Ser-CONH2

SEQ ID NO: 18 - protein, 39 aa, synthetic construct; identical to SEQ ID NO: 13
  CHAIN 1..39: note = wherein, the side chain of amino acids are free or protected with a suitable protecting group
  SITE 20: note = Lys* is a side chain present In Tirzepatide at Lys (20 position)
  SITE 13: note = Aib
  SITE 2: note = Aib
  SITE 1: note = Acety-Tyr
  SITE 39: note = Ser-CONH2

SEQ ID NO: 19 - protein, 39 aa, synthetic construct
  CHAIN 1..39: note = wherein, the side chain of amino acids are free or protected with a suitable protecting group
  SITE 20: note = Lys* is a side chain present In Tirzepatide at Lys (20 position)
  SITE 1: note = NH2-Tyr
  SITE 2: note = Aib
  SITE 39: note = Ser-CONH2
  SITE 13: note = Aib
  SITE 15: note = D-IsoAsp
YXEGTFTSDYSIXLXKIAQKAFVQWLIAGGPSSGAPPPS

SEQ ID NO: 20 - protein, 39 aa, synthetic construct; identical to SEQ ID NO: 19
  CHAIN 1..39: note = wherein, the side chain of amino acids are free or protected with a suitable protecting group
  SITE 20: note = Lys* is a side chain present In Tirzepatide at Lys (20 position)
  SITE 1: note = NH2-Tyr
  SITE 2: note = Aib
  SITE 13: note = Aib
  SITE 15: note = IsoAsp
  SITE 39: note = Ser-CONH2

SEQ ID NO: 21 - protein, 39 aa, synthetic construct; identical to SEQ ID NO: 13
  CHAIN 1..39: note = wherein, the side chain of amino acids are free or protected with a suitable protecting group
  SITE 20: note = Lys* is a side chain present In Tirzepatide at Lys (20 position)
  SITE 1: note = NH2-Tyr
  SITE 39: note = Ser-CONH2
  SITE 2: note = Aib
  SITE 13: note = Aib
  SITE 15: note = D-Asp

SEQ ID NO: 22 - protein, 39 aa, synthetic construct
  CHAIN 1..39: note = wherein, the side chain of amino acids are free or protected with a suitable protecting group
  SITE 20: note = Lys* is a side chain present In Tirzepatide at Lys (20 position)
  SITE 1: note = NH2-Tyr
  SITE 2: note = Aib
  SITE 13: note = Aib
  SITE 39: note = Ser-CONH2
YXEGTFTSDYSIXLDKIAEKAFVQWLIAGGPSSGAPPPS

SEQ ID NO: 23 - protein, 39 aa, synthetic construct
  CHAIN 1..39: note = wherein, the side chain of amino acids are free or protected with a suitable protecting group
  SITE 20: note = Lys* is a side chain present In Tirzepatide at Lys (20 position)
  SITE 1: note = NH2-Tyr
  SITE 39: note = Ser-CONH2
  SITE 2: note = Aib
  SITE 13: note = Aib
YXEGTFTSDYSIXLDKIAQKAFVEWLIAGGPSSGAPPPS

SEQ ID NO: 24 - protein, 38 aa, synthetic construct
  CHAIN 1..38: note = wherein, the side chain of amino acids are free or protected with a suitable protecting group
  SITE 20: note = Lys* is a side chain present In Tirzepatide at Lys (20 position)
  SITE 1: note = NH2-Tyr
  SITE 2: note = Aib
  SITE 13: note = Aib
  SITE 38: note = Pro-CONH2
YXEGTFTSDYSIXLDKIAQKAFVQWLIAGGPSSGAPPP

(19) United States
(12) Patent Application Publication
(10) Pub. No.: US 2025/0019410 A1
(43) Pub. Date: Jan. 16, 2025

(54) PROCESS FOR THE PREPARATION OF TIRZEPATIDE OR PHARMACEUTICALLY ACCEPTABLE SALT THEREOF

(71) Applicant: Sun Pharmaceutical Industries Ltd., MUMBAI, MAHARASHTRA (IN)

(72) Inventors: Arunkumar YADAV, Vadodara, Gujarat (IN); Kunal PANDYA, Vadodara, Gujarat (IN); Bhavna TAILOR, Vadodara, Gujarat (IN); Brijesh PATEL, Vadodara, Gujarat (IN); Jigar JOSHI, Vadodara, Gujarat (IN); Vipulkumar PATEL, Vadodara, Gujarat (IN); Mohan PRASAD, Gurugram, Haryana (IN)

(21) Appl. No.: 18/712,627

(22) PCT Filed: Nov. 22, 2022
(86) PCT No.: PCT/IB2022/061286

§ 371 Date: May 22, 2024

(30) Foreign Application Priority Data
Nov. 22, 2021 (IN) 202121053645

(51) Int. Cl. C07K 14/605; A61K 38/26; A61K 47/54

(52) U.S. Cl. CPC C07K 14/605; A61K 38/26; A61K 47/542

(57) ABSTRACT

The present invention relates to tirzepatide or a pharmaceutically acceptable salt thereof. The present invention also relates to a process for the preparation of tirzepatide or pharmaceutically acceptable salt thereof. The present invention also relates to novel fragments as intermediates and use thereof in the preparation of tirzepatide.

### PRIORITY APPLICATION

This application is a national stage of International Patent Application No. PCT/IB2022/061286, filed Nov. 22, 2022, which claims priority of to Indian Application No. IN202121053645 filed Nov. 22, 2021, the entire contents each of which are hereby incorporated by reference herein.

### FIELD OF THE INVENTION

The present invention relates to a process for the preparation of tirzepatide or a pharmaceutically acceptable salt thereof. The process according to the present invention provides novel fragments as intermediates and use thereof in the preparation of tirzepatide or a pharmaceutically acceptable salt thereof. The present invention further relates to tirzepatide or a pharmaceutically acceptable salt thereof, which is obtained by the process according to the present invention.

### BACKGROUND OF THE INVENTION

Tirzepatide is a fatty acid modified peptide with dual gastric inhibitory polypeptide (GIP) and glucagon-like peptide-1 (GLP-1) receptor agonist activity useful for the treatment of type 2 diabetes mellitus, nonalcoholic steatohepatitis (NASH) and chronic weight management. Tirzepatide is an active Phase III clinical candidate and also known as LY3298176. Tirzepatide consist of a 39 amino acid peptide backbone along with a side chain at residue 20. Out of the 39 amino acids, 37 are naturally occurring while two are modified amino acid residues present at positions 2 and 13. Tirzepatide is represented by the structure of formula A as below:

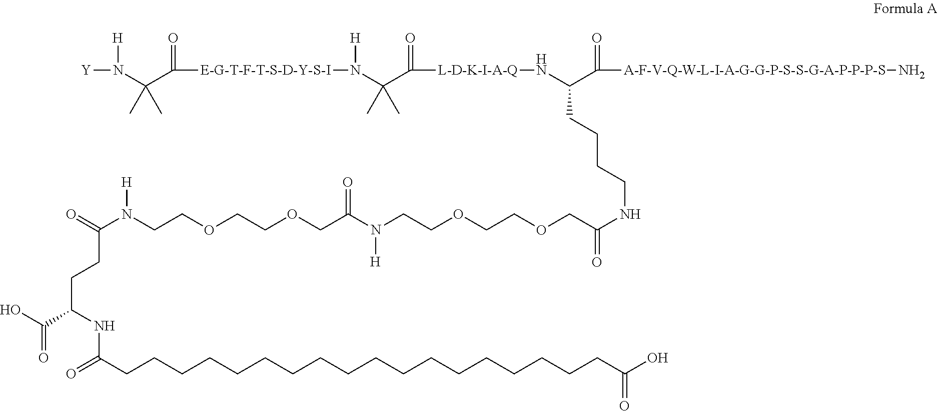

Tirzepatide was first disclosed and claimed in U.S. Pat. No. 9,474,780 (the “'780 patent”). The '780 patent describes a method for the preparation of tirzepatide trifluoroacetate salt by conventional solid phase peptide synthesis using standard Fmoc/tBu strategy and Rink Amide resin solid-phase peptide synthesis protocols in an automated peptide synthesizer. After finishing the elongation of the peptide-resin described above, the orthogonal protecting group present on Lys20 was removed to allow site-specific conjugation of the fatty acid side-chain. The resulting peptide sequence was deprotected and cleaved from the resin followed by precipitation of crude tirzepatide with cold ether. The crude tirzepatide was purified by reversed-phase HPLC followed by lyophilizing the pure fractions.

A similar approach was described in Molecular Metabolism 2018, 18, 1-12, wherein the site-specific conjugation of the fatty acid moiety to Lys20 was followed by synthesis of the linear sequence using standard Fmoc/tBu solid-phase peptide synthesis protocols in an automated peptide synthesizer.

An International Published Patent Application WO2020/159949 discloses certain peptide fragments as intermediates for the preparation of tirzepatide and a process for the preparation thereof.

Org. Process Res. Dev. 2021, 25, 1628-1636 further provides a hybrid SPPS/LPPS approach for the preparation of tirzepatide.

Another International Published Patent Application WO2021/158444 discloses a process for the preparation of tirzepatide using a solid-phase peptide synthesizer comprising at least two resin reactors in series.

A published Chinese Patent Application CN112110981 discloses a process for the preparation of the fatty-diacid side-chain using a solid-phase synthetic resin conjugate.

Another published Chinese Patent Application CN112661815 discloses a purification process of crude tirzepatide in order to remove isomer impurities.

There exists a pressing need for processes and intermediates to enable efficient production of tirzepatide having a desired purity.

The present invention describes a process for the preparation of tirzepatide or a pharmaceutically acceptable salt thereof, wherein the tirzepatide obtained by the said process has a higher purity than existing products. The present invention further describes novel fragments as intermediates and uses thereof for the preparation of tirzepatide or a pharmaceutically acceptable salt thereof.

The inventors of the present invention have surprisingly found that tirzepatide or a pharmaceutically acceptable salt thereof, as obtained according to the process of present invention possesses desirable properties when formulated as a pharmaceutical composition.

### SUMMARY OF THE INVENTION

In the first aspect, the present invention relates to a process for the preparation of tirzepatide or a pharmaceutically acceptable salt thereof, comprising use of at least one polypeptide or a pharmaceutically acceptable salt thereof selected from:

| | |
|---|---|
| Thr-Phe-Thr-Ser | SEQ ID NO. 1 |
| Asp-Tyr-Ser-Ile-Aib-Leu | SEQ ID NO. 2 |
| Ala-Gln-Lys*-Ala-Phe-Val-Gln-Trp- | SEQ ID NO. 3 |
| Leu-Ile-Ala-Gly | |
| Lys*-Ala | SEQ ID NO. 4 |
| Tyr-Aib-Glu-Gly-Thr-Phe-Thr-Ser | SEQ ID NO. 5 |
| Tyr-Aib-Glu-Gly-Thr-Phe-Thr-Ser- | SEQ ID NO. 6 |
| Asp-Tyr-Ser | |
| Ile-Aib-Leu-Asp-Lys-Ile-Ala-Gln- | SEQ ID NO. 7 |
| Lys*-Ala | |
| Lys-Ile-Ala-Gln-Lys*-Ala-Phe-Val- | SEQ ID NO. 8 |
| Gln-Trp-Leu-Ile-Ala-Gly | |
| Ile-Aib-Leu-Asp¹⁵-Lys-Ile-Ala-Gln- | SEQ ID NO. 10 |
| Lys²⁰(Side chain)-Ala-Phe-Val-Gln- | |
| Trp²⁵-Leu-Ile-Ala-Gly | |

wherein, the terminal amino acids are free, resin bound or protected with a suitable protecting group; and
wherein, the side chain of amino acids are free or protected with a suitable protecting group.

In the second aspect, the present invention relates to tirzepatide or a pharmaceutically acceptable salt thereof, obtained by following a process comprising use of at least one polypeptide or a pharmaceutically acceptable salt thereof selected from:

| | |
|---|---|
| Thr-Phe-Thr-Ser | SEQ ID NO. 1 |
| Asp-Tyr-Ser-Ile-Aib-Leu | SEQ ID NO. 2 |
| Ala-Gln-Lys*-Ala-Phe-Val-Gln-Trp- | SEQ ID NO. 3 |
| Leu-Ile-Ala-Gly | |
| Lys*-Ala | SEQ ID NO. 4 |
| Tyr-Aib-Glu-Gly-Thr-Phe-Thr-Ser | SEQ ID NO. 5 |
| Tyr-Aib-Glu-Gly-Thr-Phe-Thr-Ser- | SEQ ID NO. 6 |
| Asp-Tyr-Ser | |
| Ile-Aib-Leu-Asp-Lys-Ile-Ala-Gln- | SEQ ID NO. 7 |
| Lys*-Ala | |
| Lys-Ile-Ala-Gln-Lys*-Ala-Phe-Val- | SEQ ID NO. 8 |
| Gln-Trp-Leu-Ile-Ala-Gly | |
| Ile-Aib-Leu-Asp¹⁵-Lys-Ile-Ala-Gln- | SEQ ID NO. 10 |
| Lys²⁰(Side chain)-Ala-Phe-Val- | |
| Gln-Trp²⁵-Leu-Ile-Ala-Gly | |

wherein, the terminal amino acids are free, resin bound or protected with a suitable protecting group; and
wherein, the side chain of amino acids are free or protected with a suitable protecting group.

The present invention further relates to tirzepatide or a pharmaceutically acceptable salt thereof comprising not more than 0.5% of a single major impurity.

The present invention relates to tirzepatide or a pharmaceutically acceptable salt thereof free of a compound of SEQ ID NO. 11, SEQ ID NO. 12, SEQ ID NO. 13, SEQ ID NO. 15, SEQ ID NO. 17, SEQ ID NO. 18, SEQ ID NO. 19, SEQ ID NO. 20, SEQ ID NO. 21, SEQ ID NO. 22, SEQ ID NO. 23 or SEQ ID NO. 24 or a pharmaceutically acceptable salt thereof.

The present invention relates to the tirzepatide comprising less than 1% of a compound of SEQ ID NO. 22.

The present invention further relates to the tirzepatide comprising less than 0.5% of a compound of SEQ ID NO. 22.

The present invention relates to a compound of SEQ ID NO. 1.

The present invention relates to a compound of SEQ ID NO. 2.

The present invention relates to a compound of SEQ ID NO. 3.

The present invention relates to a compound of SEQ ID NO. 4.

The present invention relates to a compound of SEQ ID NO. 5.

The present invention relates to a compound of SEQ ID NO. 6.

The present invention relates to a compound of SEQ ID NO. 7.

The present invention relates to a compound of SEQ ID NO. 8.

The present invention relates to a compound of SEQ ID NO. 10.

The present invention relates to a compound of SEQ ID NO. 11.

The present invention relates to a compound of SEQ ID NO. 12.

The present invention relates to a compound of SEQ ID NO. 13.

The present invention relates to a compound of SEQ ID NO. 15.

The present invention relates to a compound of SEQ ID NO. 17.

The present invention relates to a compound of SEQ ID NO. 18.

The present invention relates to a compound of SEQ ID NO. 19.

The present invention relates to a compound of SEQ ID NO. 20.

The present invention relates to a compound of SEQ ID NO. 21.

The present invention relates to a compound of SEQ ID NO. 22.

The present invention relates to a compound of SEQ ID NO. 23.

The present invention relates to a compound of SEQ ID NO. 24.

The present invention relates to the compound of SEQ ID NO. 1, SEQ ID NO. 2, SEQ ID NO. 3, SEQ ID NO. 4, SEQ ID NO. 5, SEQ ID NO. 6, SEQ ID NO. 7, SEQ ID NO. 8, SEQ ID NO. 10 or a pharmaceutically acceptable salt thereof for use in the synthesis of tirzepatide or a pharmaceutically acceptable salt thereof.

The present invention relates to the sodium salt of tirzepatide.

The present invention relates to the potassium salt of tirzepatide.

The present invention relates to the ammonium salt of tirzepatide.

The present invention relates to the acetate salt of tirzepatide.

The present invention relates to tirzepatide or a pharmaceutically acceptable salt thereof substantially free from impurities selected from SEQ ID NO. 11, SEQ ID NO. 12, SEQ ID NO. 13, SEQ ID NO. 15, SEQ ID NO. 17, SEQ ID NO. 18, SEQ ID NO. 19, SEQ ID NO. 20, SEQ ID NO. 21, SEQ ID NO. 22, SEQ ID NO. 23 or SEQ ID NO. 24.

The present invention further relates to tirzepatide or a pharmaceutically acceptable salt thereof substantially free of a compound of SEQ ID NO. 22.

The present invention relates to a pharmaceutical composition consisting essentially of tirzepatide or a pharmaceutically acceptable salt thereof prepared by a process of present invention.

### Abbreviations

SPPS: Solid Phase Peptide Synthesis
LPPS: Liquid Phase Peptide Synthesis
Fmoc: Fluorenylmethyloxycarbonyl chloride
Mmt: Monomethoxytrityl
DCM: Dichloro Methane
DMF: Dimethylformamide
TFA: Trifluoroacetic acid
AEEA: 17-amino-10-oxo-3,6,12,15 tetraoxa-9-aza heptadecanoic acid

### DETAILED DESCRIPTION OF THE INVENTION

### Definitions

The terms “about” as used herein refers to as modifying a term or value such that it is not an absolute. Such terms will be defined by the circumstances and the terms that they modify as those terms are understood by those of skill in the art. This includes, at very least, the degree of expected experimental error, technique error and instrument error for a given technique used to measure a value. The term “about” when used in the present application preceding a number and referring to it, is meant to designate any value which lies within the range of ±10%, preferably within a range of ±5%.

The terms “comprising” and “comprises” as used herein are to be construed as open ended and mean the elements recited, or their equivalents in structure or function, plus any other element or elements which are not recited.

As used herein, when an amino acid abbreviation appears with a number above the amino acid, the number refers to the corresponding amino acid position in the final tirzepatide product. The numbers are provided for convenience and the appearance or absence of such numbers in a sequence does not influence the amino acid sequence or the peptide indicated in such sequence.

In the first aspect, the present invention relates to a process for the preparation of tirzepatide or a pharmaceutically acceptable salt thereof, wherein the process comprises use of at least one polypeptide or a pharmaceutically acceptable salt thereof selected from:

| | |
|---|---|
| Thr⁵-Phe-Thr-Ser⁸ | SEQ ID NO. 1 |
| Asp⁹-Tyr-Ser-Ile-Aib-Leu¹⁴ | SEQ ID NO. 2 |
| Ala¹⁸-Gln-Lys*-Ala-Phe-Val-Gln- | SEQ ID NO. 3 |
| Trp²⁵-Leu-Ile-Ala-Gly²⁹ | |
| Lys*²⁰-Ala²¹ | SEQ ID NO. 4 |
| Tyr¹-Aib-Glu-Gly-Thr-Phe-Thr-Ser8 | SEQ ID NO. 5 |
| Tyr¹-Aib-Glu-Gly-Thr-Phe-Thr-Ser- | SEQ ID NO. 6 |
| Asp-Tyr-Ser¹¹ | |
| Ile¹²-Aib-Leu-Asp-Lys-Ile-Ala-Gln- | SEQ ID NO. 7 |
| Lys*-Ala²¹ | |
| Lys¹⁶-Ile-Ala-Gln-Lys*-Ala-Phe-Val- | SEQ ID NO. 8 |
| Gln-Trp-Leu-Ile-Ala-Gly²⁹ | |
| Ile-Aib-Leu-Asp¹⁵-Lys-Ile-Ala-Gln- | SEQ ID NO. 10 |
| Lys²⁰(Side chain)-Ala-Phe-Val-Gln- | |
| Trp²⁵-Leu-Ile-Ala-Gly | |

wherein, the terminal amino acids are free, resin bound or protected with a suitable protecting group; and
wherein, the side chain of amino acids are free or protected with a suitable protecting group.

In one embodiment, the side chain of amino acids are free or protected with a suitable protecting group. In a preferred embodiment, the side chain of amino acids are protected. For example, hydroxyl groups of Ser, Tyr or Thr are protected with a tertiary butyl (tert-butyl/-tBu) group; carboxylic acid groups of Glu and Asp are protected with a tertiary butyl (tert-butyl/-tBu) group; and an amide group of Gln is protected with a trityl (-trt) group.

In another embodiment, the side chain amino (ε amino) group of Lys or Lys* is free, protected or acylated with the side chain moiety. In another embodiment, the side chain amino (ε amino) group of Lys is protected with Boc, Fmoc, Alloc, Mmt or iVDde. In yet another embodiment, the side chain amino (ε amino) group of Lys* is protected with Alloc or iVDde; or is acylated with the side chain of Moiety A or di-tertiary butyl ester of Moiety A.

In another embodiment, the amino groups of terminal amino acids in said polypeptides are free, resin bound or protected with a suitable protecting group. Depending upon the terminal amino acid, the suitable protecting group is selected from Boc, Fmoc, Alloc or iVDde.

In another embodiment, the carboxylic acid groups of terminal amino acids in said polypeptide are free, resin bound or protected with a tertiary butyl (tert-butyl/-tBu) group.

According to an embodiment, the process of the preparation of tirzepatide or a pharmaceutically acceptable salt thereof further comprises use of a polypeptide of SEQ ID NO. 9.

In one aspect, the process for the preparation of tirzepatide comprises use of polypeptides or pharmaceutically acceptable salts thereof having amino acid sequences as depicted below:

| | |
|---|---|
| Thr⁵-Phe-Thr-Ser⁸ | SEQ ID NO. 1 |
| Asp⁹-Tyr-Ser-Ile-Aib-Leu¹⁴ | SEQ ID NO. 2 |
| Ala¹⁸-Gln-Lys*-Ala-Phe-Val-Gln- | SEQ ID NO. 3 |
| Trp²⁵-Leu-Ile-Ala-Gly²⁹ | |
| Tyr¹-Aib-Glu-Gly⁴ | SEQ ID NO. 9 |

According to an aspect, the process comprises use of a polypeptide of SEQ ID NO. 3, wherein Lys* is substituted with the side chain present at Lys(20) of tirzepatide.

According to another aspect, the process comprises use of a polypeptide of SEQ ID NO. 3, wherein Lys* is protected using a protecting group. The protecting group may be selected from Boc, Fmoc, Alloc, Mmt or iVDde. According to a specific embodiment, the protecting group is iVDde.

In another aspect, the process for the preparation of tirzepatide comprises use of a polypeptide or a pharmaceutically acceptable salt thereof having an amino acid sequence of SEQ ID NO. 4.

In yet another aspect, the process for the preparation of tirzepatide comprises use of polypeptides or pharmaceutically acceptable salts thereof having amino acid sequences as depicted below:

| | |
|---|---|
| Thr⁵-Phe-Thr-Ser⁸ | SEQ ID NO. 1 |
| Asp⁹-Tyr-Ser-Ile-Aib-Leu¹⁴ | SEQ ID NO. 2 |
| Lys*²⁰-Ala²¹ | SEQ ID NO. 4 |
| Tyr¹-Aib-Glu-Gly⁴ | SEQ ID NO. 9 |

In yet another aspect, the process for the preparation of tirzepatide comprises use of polypeptides or pharmaceutically acceptable salts thereof having amino acid sequences as depicted below:

| | |
|---|---|
| Tyr¹-Aib-Glu-Gly-Thr-Phe-Thr-Ser⁸ | SEQ ID NO. 5 |
| Asp⁹-Tyr-Ser-Ile-Aib-Leu¹⁴ | SEQ ID NO. 2 |
| Lys*²⁰-Ala²¹ | SEQ ID NO. 4 |

In yet another aspect, the process for the preparation of tirzepatide, comprising use of polypeptides or pharmaceutically acceptable salts thereof having amino acid sequences as depicted below:

| | |
|---|---|
| Tyr¹-Aib-Glu-Gly-Thr-Phe-Thr-Ser- | SEQ ID NO. 6 |
| Asp-Tyr-Ser¹¹ | |
| Ile¹²-Aib-Leu-Asp-Lys-Ile-Ala-Gln- | SEQ ID NO. 7 |
| Lys*-Ala²¹ | |

In yet another aspect, the process for the preparation of tirzepatide comprises use of polypeptides or pharmaceutically acceptable salts thereof having amino acid sequences as depicted below:

| | |
|---|---|
| Tyr¹-Aib-Glu-Gly-Thr-Phe-Thr-Ser- | SEQ ID NO. 6 |
| Asp-Tyr-Ser¹¹ | |
| Lys¹⁶-Ile-Ala-Gln-Lys*-Ala-Phe-Val- | SEQ ID NO. 8 |
| Gln-Trp-Leu-Ile-Ala-Gly²⁹ | |

In one embodiment, the side chain of the amino acids are free or protected with a suitable protecting group.

According to an embodiment, the present invention relates to a compound of SEQ ID NO. 1 or a pharmaceutically acceptable salt thereof. According to an embodiment, the present invention relates to a compound of SEQ ID NO. 2 or a pharmaceutically acceptable salt thereof. According to an embodiment, the present invention relates to a compound of SEQ ID NO. 3 or a pharmaceutically acceptable salt thereof. According to an embodiment, the present invention relates to a compound of SEQ ID NO. 4 or a pharmaceutically acceptable salt thereof. According to an embodiment, the present invention relates to a compound of SEQ ID NO. 5 or a pharmaceutically acceptable salt thereof. According to an embodiment, the present invention relates to a compound of SEQ ID NO. 6 or a pharmaceutically acceptable salt thereof. According to an embodiment, the present invention relates to a compound of SEQ ID NO. 7 or a pharmaceutically acceptable salt thereof. According to an embodiment, the present invention relates to a compound of SEQ ID NO. 8 or a pharmaceutically acceptable salt thereof. According to an embodiment, the present invention relates to a compound of SEQ ID NO. 10 or a pharmaceutically acceptable salt thereof.

According to an embodiment, the side chain of amino acids of the compounds of SEQ ID NO. 1 to SEQ ID NO. 8 and SEQ ID NO. 10 are free or protected with a suitable protecting group.

According to an embodiment, the compounds of SEQ ID NO. 1 to SEQ ID NO. 8 and SEQ ID NO. 10 are prepared by the methods disclosed in the present invention.

In a preferred embodiment, the side chain of amino acids are protected. For example, hydroxyl groups of Ser, Tyr or Thr are protected with a tertiary butyl (tert-butyl/-tBu) group; carboxylic acid groups of Glu and Asp are protected with a tertiary butyl (tert-butyl/-tBu) group; and an amide group of Gln is protected with atrityl (-trt) group.

In another embodiment, the side chain amino (ε amino) group of Lys or Lys* is free, protected or acylated with the side chain moiety. In another embodiment, the side chain amino (ε amino) group of Lys is protected with Boc, Fmoc, Alloc, Mmt or iVDde. In yet another embodiment, the side chain amino (ε amino) group of Lys* is protected with Alloc or iVDde; or is acylated with the side chain of Moiety A or di-tertiary butyl ester of Moiety A.

In another embodiment, the terminal amino acids are free, resin bound or protected with a suitable protecting group.

In another embodiment, the amino groups of terminal amino acids in said polypeptides are free or protected with a suitable protecting group. Depending upon the terminal amino acid, the suitable protecting group is selected from Boc, Fmoc, Alloc or iVDde.

In another embodiment, the carboxylic acid groups of terminal amino acids in said polypeptide are free, resin bound or protected with a tertiary butyl (tert-butyl/-tBu) group.

In one embodiment, the process of the present invention comprises coupling of said polypeptides or pharmaceutically acceptable salts thereof, using solid phase peptide synthesis (SPPS), liquid phase peptide synthesis (LPPS) or a hybrid SPPS/LPPS approach.

Solid-phase peptide synthesis (SPPS) is a resin-based technology that involves the three-step repeating sequence consisting of (1) de-protection; (2) activation of an incoming amino acid; and (3) coupling of the activated amino acid to the growing peptide on resin. This sequence is repeated to synthesize the protected peptide which can then be cleaved from the resin followed by protecting group removal.

Liquid-phase peptide synthesis (LPPS) is analogous to SPPS, except the growing peptide is not bound to the resin and the C-terminus of the peptide is either a nonreactive amide or a protected ester.

In the second aspect, the present invention relates to tirzepatide or pharmaceutically acceptable salt thereof, obtained by a process comprising use of at least one polypeptide or a pharmaceutically acceptable salt thereof selected from:

| | |
|---|---|
| Thr⁵-Phe-Thr-Ser⁸ | SEQ ID NO. 1 |
| Asp⁹-Tyr-Ser-Ile-Aib-Leu¹⁴ | SEQ ID NO. 2 |
| Ala¹⁸-Gln-Lys*-Ala-Phe-Val-Gln- | SEQ ID NO. 3 |
| Trp²⁵-Leu-Ile-Ala-Gly²⁹ | |
| Lys*²⁰-Ala²¹ | SEQ ID NO. 4 |
| Tyr¹-Aib-Glu-Gly-Thr-Phe-Thr-Ser⁸ | SEQ ID NO. 5 |
| Tyr¹-Aib-Glu-Gly-Thr-Phe-Thr-Ser- | SEQ ID NO. 6 |
| Asp-Tyr-Ser¹¹ | |
| Ile¹²-Aib-Leu-Asp-Lys-Ile-Ala-Gln- | SEQ ID NO. 7 |
| Lys*-Ala²¹ | |
| Lys¹⁶-Ile-Ala-Gln-Lys*-Ala-Phe-Val- | SEQ ID NO. 8 |
| Gln-Trp-Leu-Ile-Ala-Gly²⁹ | |
| Ile-Aib-Leu-Asp¹⁵-Lys-Ile-Ala-Gln- | SEQ ID NO. 10 |
| Lys²⁰(Side chain)-Ala-Phe-Val-Gln- | |
| Trp²⁵-Leu-Ile-Ala-Gly | |

wherein, the terminal amino acids are free, resin bound or protected with a suitable protecting group; and
wherein, the side chain of amino acids are free or protected with a suitable protecting group.

In an embodiment, the present invention relates to tirzepatide or a pharmaceutically acceptable salt thereof, obtained by a comprising use of a polypeptide or pharmaceutically acceptable salt thereof selected from: SEQ ID NO. 1, SEQ ID NO. 2, SEQ ID NO. 3 and SEQ ID NO. 9.

In another embodiment, the present invention relates to tirzepatide or pharmaceutically acceptable salt thereof, obtained by a process comprising use of a polypeptide or pharmaceutically acceptable salt thereof selected from: SEQ ID NO. 1, SEQ ID NO. 2, SEQ ID NO. 4 and SEQ ID NO. 9.

In yet another embodiment, the present invention relates to tirzepatide or pharmaceutically acceptable salt thereof, obtained by a process comprising use of a polypeptide or pharmaceutically acceptable salt thereof selected from: SEQ ID NO. 5, SEQ ID NO. 2 and SEQ ID NO. 4.

In an embodiment, the present invention relates to tirzepatide or pharmaceutically acceptable salt thereof, obtained by a process comprising use of a polypeptide or pharmaceutically acceptable salt thereof selected from: SEQ ID NO. 6 and SEQ ID NO. 7.

In another embodiment, the present invention relates to tirzepatide or pharmaceutically acceptable salt thereof, obtained by a process comprising use of a polypeptide or pharmaceutically acceptable salt thereof selected from: SEQ ID NO. 6 and SEQ ID NO. 10.

In yet another embodiment, the present invention relates to tirzepatide or pharmaceutically acceptable salt thereof, obtained by a process comprising use of a polypeptide or pharmaceutically acceptable salt thereof selected from: SEQ ID NO. 6 and SEQ ID NO. 8.

In yet another embodiment, the present invention relates to tirzepatide or pharmaceutically acceptable salt thereof, obtained by a process comprising use of a polypeptide or pharmaceutically acceptable salt thereof selected from: SEQ ID NO. 4.

In one embodiment, tirzepatide or a pharmaceutically acceptable salt thereof having purity of at least about 99.0% is obtained by a process comprising use of polypeptides or pharmaceutically acceptable salts thereof selected from:

| | |
|---|---|
| Thr⁵-Phe-Thr-Ser⁸ | SEQ ID NO. 1 |
| Asp⁹-Tyr-Ser-Ile-Aib-Leu¹⁴ | SEQ ID NO. 2 |
| Ala¹⁸-Gln-Lys*-Ala-Phe-Val-Gln- | SEQ ID NO. 3 |
| Trp²⁵-Leu-Ile-Ala-Gly²⁹ | |
| Lys*²⁰-Ala²¹ | SEQ ID NO. 4 |
| Tyr¹-Aib-Glu-Gly-Thr-Phe-Thr-Ser | SEQ ID NO. 5 |
| Tyr¹-Aib-Glu-Gly-Thr-Phe-Thr-Ser- | SEQ ID NO. 6 |
| Asp-Tyr-Ser¹¹ | |
| Ile¹²-Aib-Leu-Asp-Lys-Ile-Ala-Gln- | SEQ ID NO. 7 |
| Lys*-Ala²¹ | |
| Lys¹⁶-Ile-Ala-Gln-Lys*-Ala-Phe-Val- | SEQ ID NO. 8 |
| Gln-Trp-Leu-Ile-Ala-Gly²⁹ | |
| Ile-Aib-Leu-Asp¹⁵-Lys-Ile-Ala-Gln- | SEQ ID NO. 10 |
| Lys²⁰(Side chain)-Ala-Phe-Val-Gln- | |
| Trp²⁵-Leu-Ile-Ala-Gly | |

wherein, the terminal amino acids are free, resin bound or protected with a suitable protecting group; and
wherein, the side chain of amino acids are free or protected with a suitable protecting group.

In another embodiment, tirzepatide or a pharmaceutically acceptable salt thereof having individual impurities levels below 0.15% is obtained by a process comprising use of polypeptides or pharmaceutically acceptable salts thereof selected from:

| | |
|---|---|
| Thr⁵-Phe-Thr-Ser⁸ | SEQ ID NO. 1 |
| Asp⁹-Tyr-Ser-Ile-Aib-Leu¹⁴ | SEQ ID NO. 2 |
| Ala¹⁸-Gln-Lys*-Ala-Phe-Val-Gln- | SEQ ID NO. 3 |
| Trp²⁵-Leu-Ile-Ala-Gly²⁹ | |
| Lys*²⁰-Ala²¹ | SEQ ID NO. 4 |
| Tyr¹-Aib-Glu-Gly-Thr-Phe-Thr-Ser⁸ | SEQ ID NO. 5 |
| Tyr¹-Aib-Glu-Gly-Thr-Phe-Thr-Ser- | SEQ ID NO. 6 |
| Asp-Tyr-Ser¹¹ | |
| Ile¹²-Aib-Leu-Asp-Lys-Ile-Ala-Gln- | SEQ ID NO. 7 |
| Lys*-Ala²¹ | |
| Lys¹⁶-Ile-Ala-Gln-Lys*-Ala-Phe-Val- | SEQ ID NO. 8 |
| Gln-Trp-Leu-Ile-Ala-Gly²⁹ | |
| Ile-Aib-Leu-Asp¹⁵-Lys-Ile-Ala-Gln- | SEQ ID NO. 10 |
| Lys²⁰(Side chain)-Ala-Phe-Val-Gln- | |
| Trp²⁵-Leu-Ile-Ala-Gly | |

wherein, the terminal amino acids are free, resin bound or protected with a suitable protecting group; and
wherein, the side chain of amino acids are free or protected with a suitable protecting group.

In another embodiment, the tirzepatide or pharmaceutically acceptable salts thereof prepared by the processes of the instant invention has a purity of at least about 99.0%.

In one embodiment, the tirzepatide or pharmaceutically acceptable salts thereof prepared by the processes of the instant invention have individual impurities levels below 0.15%.

In one embodiment, tirzepatide or pharmaceutically acceptable salts thereof prepared by the processes of the instant invention have individual impurities levels below 0.15%, wherein the individual impurities are selected from tirzepatide(des-side chain), Lys(20)-alpha-Glu-side chain tirzepatide, D-Ser(08)-tirzepatide, D-Tyr(01)-tirzepatide, D-Asp(15)-tirzepatide, IsoAsp(15)-tirzepatide, D-IsoAsp(15)-tirzepatide, tirzepatide-acid, Glu(24)-tirzepatide, Glu(19)-tirzepatide, Des-Tyr-tirzepatide, Des-Tyr-Aib-tirzepatide, Des-Ser(39)-tirzepatide or acetyl-tirzepatide.

According to an embodiment, the present invention relates to tirzepatide or pharmaceutically acceptable salts thereof that are substantially free of impurities.

The term ‘substantially free’ as used herein means that the individual impurities in the product are not more than 1%. According to an embodiment, tirzepatide or its pharmaceutically acceptable salt comprises less than 1%, preferably less than 0.8%, of a major impurity. According to an embodiment, tirzepatide or its pharmaceutically acceptable salt comprises less than 0.5% of a major impurity.

According to an embodiment, the major impurity may be one listed above or may be a compound of SEQ ID NO. 11 to SEQ ID NO. 24 as given below, wherein, the side chain of amino acids are free or protected with a suitable protecting group.

| SEQ ID NO | Sequence |
|---|---|
| SEQ ID NO. 11 | NH2-Glu-Gly-Thr-Phe-Thr-Ser-Asp-Tyr-Ser-Ile-Aib-Leu-Asp-Lys-Ile-Ala- |
| | Gln-Lys(Side chain)-Ala-Phe-Val-Gln-Trp-Leu-Ile-Ala-Gly-Gly-Pro-Ser- |
| | Ser-Gly-Ala-Pro-Pro-Pro-Ser-CONH2 |
| SEQ ID NO. 12 | NH2--Aib-Glu-Gly-Thr-Phe-Thr-Ser-Asp-Tyr-Ser-Ile-Aib-Leu-Asp-Lys-Ile- |
| | Ala-Gln-Lys(Side chain)-Ala-Phe-Val-Gln-Trp-Leu-Ile-Ala-Gly-Gly-Pro- |
| | Ser-Ser-Gly-Ala-Pro-Pro-Pro-Ser-CONH2 |
| SEQ ID NO. 13 | NH2-D-Tyr-Aib-Glu-Gly-Thr-Phe-Thr-Ser-Asp-Tyr-Ser-Ile-Aib-Leu-Asp- |
| | Lys-Ile-Ala-Gln-Lys(Side chain)-Ala-Phe-Val-Gln-Trp-Leu-Ile-Ala-Gly- |
| | Gly-Pro-Ser-Ser-Gly-Ala-Pro-Pro-Pro-Ser-CONH2 |
| SEQ ID NO. 14 | NH2-Tyr-Aib-Glu-Gly-Thr-Phe-Thr-D-Ser-Asp-Tyr-Ser-Ile-Aib-Leu-Asp- |
| | Lys-Ile-Ala-Gln-Lys(Side chain)-Ala-Phe-Val-Gln-Trp-Leu-Ile-Ala-Gly- |
| | Gly-Pro-Ser-Ser-Gly-Ala-Pro-Pro-Pro-Ser-CONH2 |
| SEQ ID NO. 15 | NH2-Tyr-Aib-Glu-Gly-Thr-Phe-Thr-Ser-Asp-Tyr-Ser-Ile-Aib-Leu-Asp-Lys- |
| | Ile-Ala-Gln-Lys(alpha-Glu-Side chain)-OH-Ala-Phe-Val-Gln-Trp-Leu-Ile- |
| | Ala-Gly-Gly-Pro-Ser-Ser-Gly-Ala-Pro-Pro-Pro-Ser-CONH2 |
| SEQ ID NO. 16 | NH2-Tyr-Aib-Glu-Gly-Thr-Phe-Thr-Ser-Asp-Tyr-Ser-Ile-Aib-Leu-Asp-Lys- |
| | Ile-Ala-Gln-Lys-Ala-Phe-Val-Gln-Trp-Leu-Ile-Ala-Gly-Gly-Pro-Ser-Ser- |
| | Gly-Ala-Pro-Pro-Pro-Ser-CONH2 |
| SEQ ID NO. 17 | NH2-Tyr-Aib-Glu-Gly-Thr-Phe-Thr-Ser-Asp-Tyr-Ser-Ile-Aib-Leu-Asp-Lys- |
| | Ile-Ala-Gln-Lys(ivdde)-Ala-Phe-Val-Gln-Trp-Leu-Ile-Ala-Gly-Gly-Pro- |
| | Ser-Ser-Gly-Ala-Pro-Pro-Pro-Ser-CONH2 |
| SEQ ID NO. 18 | Acety-Tyr-Aib-Glu-Gly-Thr-Phe-Thr-Ser-Asp-Tyr-Ser-Ile-Aib-Leu-Asp-Lys- |
| | Ile-Ala-Gln-Lys(Side chain)-Ala-Phe-Val-Gln-Trp-Leu-Ile-Ala-Gly-Gly- |
| | Pro-Ser-Ser-Gly-Ala-Pro-Pro-Pro-Ser-CONH2 |
| SEQ ID NO. 19 | NH2-Tyr-Aib-Glu-Gly-Thr-Phe-Thr-Ser-Asp-Tyr-Ser-Ile-Aib-Leu-D-IsoAsp- |
| | Lys-Ile-Ala-Gln-Lys(Side chain)-Ala-Phe-Val-Gln-Trp-Leu-Ile-Ala-Gly- |
| | Gly-Pro-Ser-Ser-Gly-Ala-Pro-Pro-Pro-Ser-CONH2 |
| SEQ ID NO. 20 | NH2-Tyr-Aib-Glu-Gly-Thr-Phe-Thr-Ser-Asp-Tyr-Ser-Ile-Aib-Leu-IsoAsp- |
| | Lys-Ile-Ala-Gln-Lys(Side chain)-Ala-Phe-Val-Gln-Trp-Leu-Ile-Ala-Gly- |
| | Gly-Pro-Ser-Ser-Gly-Ala-Pro-Pro-Pro-Ser-CONH2 |
| SEQ ID NO. 21 | NH2-Tyr-Aib-Glu-Gly-Thr-Phe-Thr-Ser-Asp-Tyr-Ser-Ile-Aib-Leu-D-Asp- |
| | Lys-Ile-Ala-Gln-Lys(Side chain)-Ala-Phe-Val-Gln-Trp-Leu-Ile-Ala-Gly- |
| | Gly-Pro-Ser-Ser-Gly-Ala-Pro-Pro-Pro-Ser-CONH2 |
| SEQ ID NO. 22 | NH2-Tyr-Aib-Glu-Gly-Thr-Phe-Thr-Ser-Asp-Tyr-Ser-Ile-Aib-Leu-Asp-Lys- |
| | Ile-Ala-Glu-Lys(Side chain)-Ala-Phe-Val-Gln-Trp-Leu-Ile-Ala-Gly-Gly- |
| | Pro-Ser-Ser-Gly-Ala-Pro-Pro-Pro-Ser-CONH2 |
| SEQ ID NO. 23 | NH2-Tyr-Aib-Glu-Gly-Thr-Phe-Thr-Ser-Asp-Tyr-Ser-Ile-Aib-Leu-Asp-Lys- |
| | Ile-Ala-Gln-Lys(Side chain)-Ala-Phe-Val-Glu-Trp-Leu-Ile-Ala-Gly-Gly- |
| | Pro-Ser-Ser-Gly-Ala-Pro-Pro-Pro-Ser-CONH2 |
| SEQ ID NO. 24 | NH2-Tyr-Aib-Glu-Gly-Thr-Phe-Thr-Ser-Asp-Tyr-Ser-Ile-Aib-Leu-Asp-Lys- |
| | Ile-Ala-Gln-Lys(Side chain)-Ala-Phe-Val-Gln-Trp-Leu-Ile-Ala-Gly-Gly- |
| | Pro-Ser-Ser-Gly-Ala-Pro-Pro-Pro-CONH2 |

According to an embodiment, the present invention provides tirzepatide or a pharmaceutically acceptable salt comprising less than 1% of a compound of SEQ ID NO. 22. According to another embodiment, the present invention provides tirzepatide or a pharmaceutically acceptable salt comprising less than 0.5% of a compound of SEQ ID NO. 22.

The processes of the present invention yielded tirzepatide with high purity. According to an embodiment, tirzepatide prepared by the processes of the present invention is more than 98% pure. According to another embodiment, tirzepatide prepared by the processes of the present invention is more than 99% pure. According to yet another embodiment, tirzepatide prepared by the processes of the present invention is more than 99.5% pure.

The processes comprising SEQ ID NO. 1 to 10, resulted in tirzepatide comprising less than 1%, particularly less than 0.5%, of a single major impurity of a compound of SEQ ID NO. 11 to SEQ ID NO. 24. Table 1 below shows the purity of tirzepatide prepared by the processes comprising the use of SEQ ID NO. 1 to 10 and the percentage of impurities present.

TABLE 1
Percentage of each impurity present in Tirzepatide

| Compounds | Ex. 9 | Ex. 10 | Ex. 11 | Ex. 12 | Ex. 13 | Ex. 14 |
|---|---|---|---|---|---|---|
| Tirzepatide | 98.82 | 99.43 | 99.47 | 99.13 | 99.46 | 99.74 |
| SEQ ID NO. 16 | ND | ND | ND | ND | ND | ND |
| SEQ ID NO. 19 | 0.03 | ND | 0.02 | 0.03 | 0.02 | 0.03 |
| SEQ ID NO. 20 | 0.01 | 0.02 | 0.02 | 0.02 | 0.01 | ND |
| SEQ ID NO. 12 | 0.17 | 0.11 | 0.13 | 0.10 | 0.09 | 0.04 |
| SEQ ID NO. 11 | 0.02 | 0.01 | ND | 0.01 | 0.01 | ND |
| SEQ ID NO. 14 | 0.29 | 0.12 | 0.10 | 0.19 | 0.15 | 0.07 |
| SEQ ID NO. 15 | ND | ND | ND | 0.01 | 0.01 | ND |
| SEQ ID NO. 21 | 0.01 | ND | ND | 0.01 | 0.01 | ND |
| SEQ ID NO. 13 | 0.01 | 0.03 | 0.04 | 0.02 | 0.01 | 0.03 |
| SEQ ID NO. 22 | ND | ND | ND | ND | ND | ND |
| SEQ ID NO. 24 | ND | 0.03 | 0.04 | 0.01 | ND | ND |
| SEQ ID NO. 23 | 0.21 | 0.07 | 0.06 | 0.14 | 0.06 | 0.03 |
| SEQ ID NO. 18 | ND | ND | ND | ND | ND | ND |
| Major Unknown | 0.21 | 0.10 | 0.07 | 0.20 | 0.08 | 0.04 |

The present invention further relates to the compounds of SEQ ID NO. 11, SEQ ID NO. 12, SEQ ID NO. 13, SEQ ID NO. 15, SEQ ID NO. 17, SEQ ID NO. 18, SEQ ID NO. 19, SEQ ID NO. 20, SEQ ID NO. 21, SEQ ID NO. 22, SEQ ID NO. 23, and SEQ ID NO. 24.

The compounds of SEQ ID NO. 11, SEQ ID NO. 12, SEQ ID NO. 13, SEQ ID NO. 15, SEQ ID NO. 17, SEQ ID NO. 18, SEQ ID NO. 19, SEQ ID NO. 20, SEQ ID NO. 21, SEQ ID NO. 22, SEQ ID NO. 23 and SEQ ID NO. 24 were analysed using mass as given in the Table 2 below:

TABLE 2
Impurity Sequence and Mass analysis

| | | Mass m/z |
|---|---|---|
| SEQ ID NO | Mol. wt | (M + 4H) + 4 |
| SEQ ID NO. 11 | 4565.22 | 1142.85 |
| SEQ ID NO. 12 | 4650.32 | 1164.12 |
| SEQ ID NO. 13 | 4813.42 | 1205.00 |
| SEQ ID NO. 14 | 4813.42 | 1205.12 |
| SEQ ID NO. 15 | 4813.42 | 1204.98 |
| SEQ ID NO. 16 | 4069.52 | 1018.78 |
| SEQ ID NO. 17 | 4275.82 | 1070.39 |
| SEQ ID NO. 18 | 4855.52 | 1215.39 |
| SEQ ID NO. 19 | 4813.42 | 1204.42 |
| SEQ ID NO. 20 | 4813.42 | 1204.22 |
| SEQ ID NO. 21 | 4813.42 | 1204.2 |
| SEQ ID NO. 22 | 4814.42 | 1204.67 |
| SEQ ID NO. 23 | 4814.42 | 1204.58 |
| SEQ ID NO. 24 | 4727.42 | 1182.5 |

According to an embodiment, the present invention relates to pharmaceutically acceptable salts of tirzepatide.

According to another embodiment, the present invention relates to a process of preparation of the pharmaceutically acceptable salts of tirzepatide.

According to an embodiment, the present invention relates to the sodium salt of tirzepatide. According to an embodiment, the present invention relates to the potassium salt of tirzepatide. According to an embodiment, the present invention relates to the ammonium salt of tirzepatide. According to an embodiment, the present invention relates to the acetate salt of tirzepatide.

In another embodiment, tirzepatide obtained according to a process of present invention is a sodium salt of tirzepatide. In an alternate aspect, tirzepatide obtained according to a process of present invention is a potassium salt of tirzepatide. In an alternate aspect, tirzepatide obtained according to a process of present invention is an ammonium salt of tirzepatide. In an alternate aspect, tirzepatide obtained according to a process of present invention is an acetate salt of tirzepatide.

According to an embodiment, the sodium content in the sodium salt of tirzepatide is from 1.5% to 2.5%. The potassium content in the potassium salt of tirzepatide is above 2.5%. The metal ion content for the present invention was determined by using Ion chromatography (Make: Thermo Fisher).

In one embodiment, the processes of the present invention yielded higher purity and more stable salts of tirzepatide. The solubility of the salts in water (see Table 3), methanol (see Table 4) and phosphate buffer (see Table 5) were tested. It was found that although all salts were considerable soluble and stable, sodium salts showed exceptionally better solubility as compared to the other salts.

TABLE 3
Solubility in Water:

| Compound | Solubility - in Water |
|---|---|
| Acetic acid content - 1.06% | Practically insoluble in water |
| Sodium content- 1.90% | Freely Soluble in water(about 100 mg/mL) |
| Sodium content- 2.12% | Freely Soluble in water(about 100 mg/mL) |
| Potassium content- 2.89% | slightly Soluble in water(about 1 mg/mL) |
| Ammonium content- 0.25% | practically insoluble in water |

TABLE 4
Solubility in Methanol:

| Compound | Solubility - in methanol |
|---|---|
| Acetic acid content - 1.06% | Freely soluble in Methanol (about 100 mg/mL) |
| Sodium content- 1.90% | Freely Soluble in Methanol(about 100 mg/mL) |
| Sodium content- 2.12% | Freely Soluble in Methanol(about 100 mg/mL) |
| Potassium content- 2.89% | slightly Soluble in methanol(about 1 mg/mL) |
| Ammonium content- 0.25% | Freely Soluble in Methanol(about 100 mg/mL) |

TABLE 5
Solubility in Phosphate buffer:

| Compound | Solubility - in Phosphate buffer pH 7 |
|---|---|
| Acetic acid content - 1.06% | Very slightly soluble in Phosphate buffer pH 7 (about 0.1 |
| | mg/mL) |
| Sodium content- 1.90% | Freely soluble in Phosphate buffer pH 7 (about 100 mg/mL) |
| Sodium content- 2.12% | Freely soluble in Phosphate buffer pH 7 (about 100 mg/mL) |
| Potassium content- 2.89% | slightly soluble in Phosphate buffer pH 7 (about 1 mg/mL) |
| Ammonium content- 0.25% | very slightly soluble in Phosphate buffer pH 7 (about 0.1 |
| | mg/mL) |

The present invention relates to a pharmaceutical composition comprising tirzepatide or a pharmaceutically acceptable salt disclosed in present invention. The composition may further comprise water or a buffer.

The present invention may involve one or more embodiments. It is to be understood that the embodiments below are illustrative of the present invention and are not intended to limit the claims to the specific embodiments exemplified. It is also to be understood that the embodiments defined herein may be used independently or in conjunction with any definition, any other embodiment defined herein. Thus, the invention contemplates all possible combinations and permutations of the various independently described embodiments.

### EXAMPLES

Instruments and analytical methods: Instruments used for characterization and analysis of the compounds of the present invention are HPLC (Waters e2695 Alliance; Detector Waters (2489 UV/Visible)).

HPLC: Waters e2695 Alliance; Detector: Acquity-QDa.

The final compounds of the present disclosure were purified by preparative HPLC procedure as outlined below:

Preparative HPLC: WATERS 2555 Quaternary gradient module (Max Total Flow: 300 mL/min, Max Pressure: 3000 psi) or Shimadzu LC-8A (Max Total Flow: 150 mL, Max Pressure: 30 Mpa), Column: Phenyl, 10μ Flow: 75 mL/min

### Mobile Phase:

| | pH 8.0 | | pH 8.2 | | pH 2.5 |
|---|---|---|---|---|---|
| Mobile | Phosphate | 1% Acetic | Ammonium | 0.1% TFA in | KH2PO4 and |
| Phase A | buffer | acid in water | formate buffer | Water | TEA in Water |
| Mobile | Acetonitrile | 1% Acetic acid in | Acetonitrile | 0.1% TFA in | Acetonitrile |
| Phase B | | Acetonitrile:n- | | Acetonitrile | |
| | | Propanol (50:50) | | | |
| Gradient | 15 to 45% | 20 to 50% | 20 to 50% | 20 to 50% | 20 to 50% |
| | Mobile Phase- | Mobile Phase- | Mobile Phase- | Mobile Phase- | Mobile Phase- |
| | B in 300 min | B in 300 min | B in 300 min | B in 300 min | B in 300 min |

The purity of the compounds of the present disclosure were analyzed by one of the RP-HPLC methods as outlined below:

### HPLC Method A:

Column: Xbridge Peptide BEH C18 (4.6 mm×250 mm, 3.5μ)
Eluent: Mobile Phase A: Buffer: Acetonitrile (900:100)
Mobile phase B: Buffer: Acetonitrile (300:700)
Buffer: Potassium dihydrogen orthophosphate in water, pH adjusted to 3.0±0.1 with
orthophosphoric acid. Flow rate: 0.8 mL/min. Detection: UV detection at 210 nm
Column Temperature: 65° C. Sample Tray temperature: 5° C. Run Time: 90 min.

| Time (min) | Mobile Phase A % | Mobile Phase B % |
|---|---|---|
| 0 | 55 | 45 |
| 3 | 55 | 45 |
| 5 | 40 | 60 |
| 60 | 39 | 61 |
| 65 | 0 | 100 |
| 75 | 0 | 100 |
| 75.01 | 55 | 45 |
| 90 | 55 | 45 |

### Method of Preparation:

### Example 1: Preparation of Compound I—Moiety A—di-tert-butyl ester

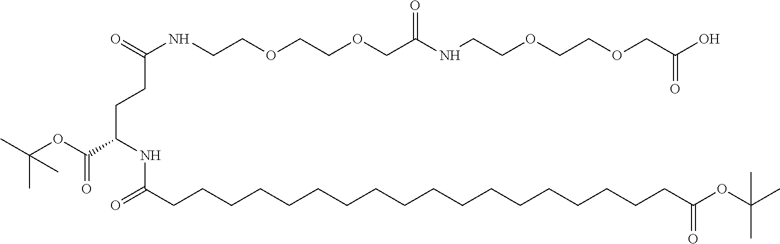 [SMILES: CC(C)(C)OC(=O)CCCCCCCCCCCCCCCCCCC(=O)N[C@@H](CCC(=O)NCCOCCOCC(=O)NCCOCCOCC(=O)O)C(=O)OC(C)(C)C]

### Moiety A—Di-Tert-Butyl Ester

Moiety A—di-tert-butyl ester was prepared using solid phase synthesis using 2-chlorotrityl chloride resin as schematically represented below. 2-[2-(2-Fmoc-aminoethoxy)ethoxy]acetic acid was attached to 2-chlorotrityl chloride resin in the presence of N,N-diisopropylethylamine to yield 2-[2-(2-Fmoc-aminoethoxy)ethoxy]acetic acid-2-Cl-Trt-Resin. The Fmoc protecting group was removed by selective de-blocking of the amino group using piperidine followed by coupling with 2-[2-(2-Fmoc-aminoethoxy)ethoxy]acetic acid in THE using a Coupling reagent/Auxiliary nucleophile/Base which yielded {(Fmoc-amino-ethoxy)-ethoxy}-acetyl-{(-amino-ethoxy)-ethoxy}-acetic acid-2-Cl-Trt-Resin. The Fmoc group was removed by selective de-blocking using piperidine and the free amino group was coupled with Fmoc-Glu-OtBu using Coupling reagent/Auxiliary nucleophile/Base to yield Fmoc-Glu({(amino-ethoxy)-ethoxy}-acetyl-{(-amino-ethoxy)-ethoxy}-aceticacid-2-Cl-Trt-Resin)-OtBu. The Fmoc group of the resultant compound was selectively de-blocked using piperidine and the free amino group was then coupled with 20-(tert-butoxy)-20-oxoicosanoic acid to give 2-[2-[2-[[2-[2-[2-[[5-tert-butoxy-4-[(20-tert-butoxy-20-oxo-icosanoyl)amino]-5-oxo-entanoyl]amino]ethoxy]ethoxy]acetyl]amino]ethoxy]ethoxy]acetic acid-2-Cl-Trt-Resin.

The intermediate was then cleaved from 2-Cl-Trt-Resin using trifluoroethanol: DCM (1:1) or 0.1% TFA in DCM or a dilute hydrochloride solution in DCM to obtain Moiety A—di-tert-butyl ester.

### Example 2: Preparation of Compound II: Fmoc-Lys(tBu-OOC-C18-CO-Glu(AEEA-AEEA)-OtBu)-OH

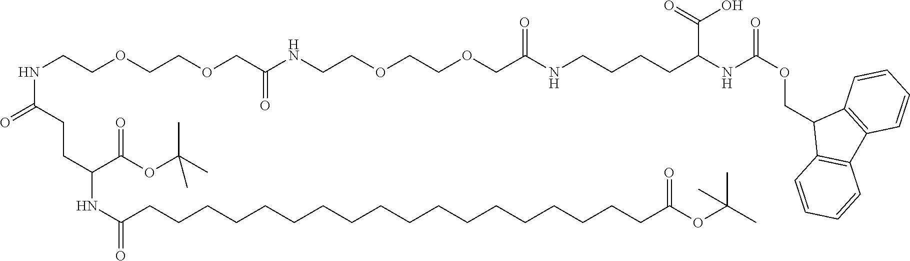 [SMILES: CC(C)(C)OC(=O)CCCCCCCCCCCCCCCCCCC(=O)NC(CCC(=O)NCCOCCOCC(=O)NCCOCCOCC(=O)NCCCCC(NC(=O)OCC1c2ccccc2-c2ccccc21)C(=O)O)C(=O)OC(C)(C)C]

### Fmoc-Lys-Moiety A—Di-Tert-Butyl Ester

### Method A:

Compound II is synthesized in solution phase. Synthesis was started using Boc protection of 2-[2-(2-aminoethoxy)ethoxy]acetic acid] followed by activation of the carboxyl group and selectively coupled with second 2-[2-(2-aminoethoxy)ethoxy]acetic acid] to get Boc-AEEA-AEEA-OH. The carboxyl group of this in-situ intermediate was further activated and selectively coupled with Fmoc-Lys-OH·HCl to get Fmoc-Lys(Boc-AEEA-AEEA-OH). The Boc group was removed using TFA to get Fmoc-Lys(NH₂-AEEA-AEEA-OH) (Intermediate-I). 20-(tert-Butoxy)-20-oxoicosanoic acid was activated and coupled with H-Glu-OtBu using a Coupling reagent/Auxiliary nucleophile/Base, which was further converted to get tBu-OOC-C18-CO-Glu(OSU)-OtBu (Intermediate-II). Intermediate-I and Intermediate-II were coupled in the presence of base to get Compound II, which was further purified by chromatographic technique to get the pure material.

### Method B:

Compound I was synthesized in accordance with the method described in Example 1 using solid phase peptide synthesis. Compound I was coupled with Fmoc-Lys-OH·HCl using Coupling reagent/Auxiliary nucleophile/Base. The reaction was monitor by TLC/HPLC followed by an aqueous/organic work-up to get Compound II. If required, the compound was further purified using a suitable chromatographic technique to get pure semisolid material.

### Example 3: Preparation of Tirzepatide—Method a (Continuous Preparation from AA 39 to AA 1)

The parent peptide was synthesized by solid-phase method. The starting resin used for synthesis was Fmoc-Rink amide resin. Selective de-blocking of the Fmoc protected amino group of rink amide resin was carried out using piperidine to yield Rink amide resin which was then coupled with Fmoc-Ser(tBu)-OH to yield Fmoc-Ser(tBu)-Rink amide Resin. This coupling reaction was performed by using Coupling reagent/Auxiliary nucleophile/Base. This completed one cycle. Acetic anhydride/Acetyl chloride and diisopropylethyl amine/pyridine was used to terminate/cap the uncoupled amino groups at every amino acid coupling. Selective de-blocking of the amino group of Fmoc-Ser(tBu)-Rink amide Resin was done using Piperidine/DMF. Then coupling with Fmoc-Pro-OH using Coupling reagent/Auxiliary nucleophile/Base provided Fmoc-Pro-Ser(tBu)-rink amide Resin. This completed the second cycle. Acetic anhydride/Acetyl Chloride and diisopropylethyl amine/pyridine were used to terminate the uncoupled amino groups at every amino acid coupling.

The above three steps, i.e., selective capping, deblocking of Fmoc-protection of amino acid attached to the resin and coupling of next amino acid residue in sequence with Fmoc-protected amino group, were repeated for the remaining 37 amino acid residues. The selective acylation, i.e., capping of uncoupled amino group done by using acetic anhydride/Acetyl chloride and diisopropylethylamine/pyridine, deprotection of Fmoc group was done using Piperidine/DMF and coupling with next Fmoc protected amino acid was done using Coupling reagent/Auxiliary nucleophile/Base. The side chain of the Fmoc-protected amino acids were protected orthogonally, e.g., hydroxyl groups of Serine, Tyrosine or Threonine were protected with tert-butyl(-tBu) group, the amino group of Lysine was protected with a tert-butyloxycarbonyl (-Boc) group and (4,4-dimethyl-2,6-dioxocyclohex-1-ylidene)-3-methylbutyl (IVDde) group, respectively, and carboxylic acid groups of aspartic acid or glutamic acid were protected with -tBu groups and an amide group of glutamine was protected with a trityl (-Trt) group. The above mentioned three steps, i.e., selective capping, deblocking and then coupling with the next Fmoc protected amino acid, were performed to get Fmoc-Tyr(tBu)-Aib-Glu(OtBu)-Gly-Thr(tBu)-Phe-Thr(tBu)-Ser(tBu)-Asp(OtBu)-Tyr(tBu)-Ser(tBu)-Ile-Aib-Leu-Asp(OtBu)-Lys(Boc)-Ile-Ala-Gln(Trt)-Lys(IVDde)-Ala-Phe-Val-Gln(Trt)-Trp-Leu-Ile-Ala-Gly-Gly- Pro-Ser(tBu)-Ser(tBu)-Gly-Ala-Pro-Pro-Pro-Ser(tBu)-resin.

De-blocking of Fmoc-Tyr(tBu)-Aib-Glu(OtBu)-Gly-Thr(tBu)-Phe-Thr(tBu)-Ser(tBu)-Asp(OtBu)-Tyr(tBu)-Ser(tBu)-Ile-Aib-Leu-Asp(OtBu)-Lys(Boc)-Ile-Ala-Gln(Trt)-Lys(IVDde)-Ala-Phe-Val-Gln(Trt)-Trp-Leu-Ile-Ala-Gly-Gly-Pro-Ser(tBu)- Ser(tBu)-Gly-Ala-Pro-Pro-Pro-Ser(tBu)-resin was carried out using piperidine followed by Boc protection of peptide resin using Boc anhydride to yield Boc-Tyr(tBu)-Aib-Glu(OtBu)-Gly-Thr(tBu)-Phe-Thr(tBu)-Ser(tBu)-Asp(OtBu)-Tyr(tBu)-Ser(tBu)-Ile-Aib-Leu-Asp(OtBu)-Lys(Boc)-Ile-Ala-Gln(Trt)-Lys(IVDde)-Ala-Phe-Val-Gln(Trt)-Trp-Leu-Ile- Ala-Gly-Gly-Pro-Ser(tBu)-Ser(tBu)-Gly-Ala-Pro-Pro-Pro-Ser(tBu)-resin. De-protection of the IVDde group of the peptide resin was carried out using hydrazine hydrate and then it was coupled with Compound I-di-tert-butyl ester using Coupling reagent/Auxiliary nucleophile/Base to yield an intermediate compound resin, Boc-Tyr(tBu)-Aib-Glu(OtBu)-Gly-Thr(tBu)-Phe-Thr(tBu)-Ser(tBu)-Asp(OtBu)-Tyr(tBu)-Ser(tBu)-Ile-Aib-Leu-Asp(OtBu)-Lys(Boc)-Ile-Ala-Gln(Trt)-Lys(NH-Moiety A di-tert-butyl ester)-Ala-Phe-Val-Gln(Trt)-Trp-Leu-Ile-Ala-Gly-Gly-Pro-Ser(tBu)-Ser(tBu)-Gly-Ala-Pro-Pro-Pro-Ser(tBu)-resin, which on cleavage and de-protection using trifluoroacetic acid with Scavenger gave crude tirzepatide. The crude product was further purified through preparative HPLC to provide pure Tirzepatide.

### Example 4: Preparation of Tirzepatide—Method B

The parent peptide was synthesized by solid-phase method. The starting resin used for synthesis was Fmoc-Rink amide resin. Selective de-blocking of Fmoc protected amino group of rink amide resin was carried out using piperidine to yield Rink amide resin which was then coupled with Fmoc-Ser(tBu)-OH to yield Fmoc-Ser(tBu)-Rink amide Resin. This coupling reaction was performed by using Coupling reagent/Auxiliary nucleophile/Base. This completed one cycle. Acetic anhydride/Acetyl chloride and diisopropylethyl amine/pyridine was used to terminate/cap the uncoupled amino groups at every amino acid coupling. Selective de-blocking of the amino group of Fmoc-Ser(tBu)-Rink amide Resin was done using piperidine/DMF. Then coupling with Fmoc-Pro-OH using Coupling reagent/Auxiliary nucleophile/Base to get Fmoc-Pro-Ser(tBu)-rink amide Resin. This completed the second cycle. Acetic anhydride/Acetyl Chloride and diisopropylethyl amine/pyridine were used to terminate the uncoupled amino groups at every amino acid coupling.

The above three steps, i.e., selective capping, deblocking of Fmoc-protection of amino acid attached to the resin and coupling of next amino acid residue in sequence with Fmoc-protected amino group, were repeated for the remaining 37 amino acid residues. The selective acylation, i.e., capping of uncoupled amino group done by using acetic anhydride/acetyl chloride and diisopropylethylamine/pyridine, deprotection of Fmoc group was done using piperidine/DMF and coupling with next Fmoc protected amino acid was done using Coupling reagent/Auxiliary nucleophile/Base. The side chain of the Fmoc-protected amino acids were protected orthogonally, e.g., hydroxyl groups of Serine, Tyrosine or Threonine were protected with tert-butyl(-tBu) groups, amino groups of Lysine was protected with tert-butyloxycarbonyl (-Boc) and Methoxytrityl (Mmt) groups, respectively, carboxylic acid groups of aspartic acid or glutamic acid were protected with -tBu groups and the amide group of glutamine was protected with a trityl (-Trt) group. The above mentioned three steps, i.e., selective capping, deblocking and then coupling with next Fmoc protected amino acid, were performed to get Fmoc-Lys(Mmt)-Ala-Phe-Val-Gln(Trt)-Trp-Leu-Ile-Ala-Gly-Gly-Pro-Ser(tBu)-Ser(tBu)-Gly-Ala-Pro-Pro-Pro-Ser(tBu)-resin.

Selectively deblocking the methoxytrityl group using TFA/DCM/AcOH or 0.1% TFA in DCM or dilute HCL in DCM was done and then it was coupled with Compound I-di-tert-butyl ester using Coupling reagent/Auxiliary nucleophile/Base to yield an intermediate compound resin, Fmoc-Lys((NH-Compound I-di-tert-butyl ester)-Ala-Phe-Val-Gln(Trt)-Trp-Leu-Ile-Ala-Gly-Gly-Pro-Ser(tBu)-Ser(tBu)-Gly-Ala-Pro-Pro-Pro-Ser(tBu)-resin. The remaining protected amino acids were coupled as per sequence using Coupling reagent/Auxiliary nucleophile/Base, followed by the capping, deblocking cycle to get intermediate, Fmoc-Tyr(tBu)-Aib-Glu(OtBu)-Gly-Thr(tBu)-Phe-Thr(tBu)-Ser(tBu)-Asp(OtBu)-Tyr(tBu)-Ser(tBu)-Ile-Aib-Leu-Asp(OtBu)-Lys(Boc)-Ile-Ala-Gln(Trt)-Lys(NH-Moiety A di-tert-butyl ester)-Ala-Phe-Val-Gln(Trt)-Trp-Leu-Ile-Ala-Gly-Gly-Pro-Ser(tBu)-Ser(tBu)-Gly-Ala-Pro-Pro-Pro-Ser(tBu)-resin.

De-blocking of the Fmoc-Tyr(tBu)-Aib-Glu(OtBu)-Gly-Thr(tBu)-Phe-Thr(tBu)-Ser(tBu)-Asp(OtBu)-Tyr(tBu)-Ser(tBu)-Ile-Aib-Leu-Asp(OtBu)-Lys(Boc)-Ile-Ala-Gln(Trt)-Lys(NH-Moiety A di-tert-butyl ester)-)-Ala-Phe-Val-Gln(Trt)-Trp-Leu-Ile-Ala-Gly-Gly-Pro-Ser(tBu)-Ser(tBu)-Gly-Ala-Pro-Pro-Pro-Ser(tBu)-resin was carried out using piperidine, followed by cleavage and de-protection using trifluoroacetic acid with a scavenger to get crude product, which was further purified through preparative HPLC to provide tirzepatide.

### Example 4: Preparation of Tirzepatide—Method C

The parent peptide was synthesized by solid-phase method. The starting resin used for synthesis was Fmoc-Rink amide resin. Selective de-blocking of Fmoc protected amino group of rink amide resin was carried out using piperidine to yield Rink amide resin which was then coupled with Fmoc-Ser(tBu)-OH to yield Fmoc-Ser(tBu)-Rink amide Resin. This coupling reaction was performed by using Coupling reagent/Auxiliary nucleophile/Base. This completed one cycle. Acetic anhydride/Acetyl chloride and diisopropylethyl amine/pyridine was used to terminate/cap the uncoupled amino groups at every amino acid coupling. Selective de-blocking of the amino group of Fmoc-Ser(tBu)-Rink amide Resin was done using piperidine/DMF and then coupling with Fmoc-Pro-OH using Coupling reagent/Auxiliary nucleophile/Base Fmoc-Pro-Ser(tBu)-rink amide Resin was done. This completed the second cycle. Acetic anhydride/acetyl chloride and diisopropylethyl amine/pyridine were used to terminate the uncoupled amino groups at every amino acid coupling.

The above three steps, i.e., selective capping, deblocking of Fmoc-protection of amino acid attached to the resin and coupling of next amino acid residue in sequence with Fmoc-protected amino group, were repeated for the remaining 37 amino acid residues. The selective acylation, i.e., capping of uncoupled amino group done by using acetic anhydride/acetyl chloride and diisopropylethylamine/pyridine, deprotection of Fmoc group was done using piperidine/DMF and coupling with next Fmoc protected amino acid was done using Coupling reagent/Auxiliary nucleophile/Base. The side chain of the Fmoc-protected amino acids were protected orthogonally, e.g., hydroxyl groups of Serine, Tyrosine or Threonine were protected with tert-butyl(-tBu) groups, the amino group of Lysine was protected with a tert-butyloxycarbonyl (-Boc) group and carboxylic acid groups of aspartic acid or glutamic acid were protected with -tBu groups and the amide group of glutamine was protected with a trityl (-Trt) group. Lysine at residue number 20 was already modifier at a side chain (Compound-II). The above mentioned three steps, i.e., selective capping, deblocking and then coupling with next Fmoc protected amino acid, were performed to get Fmoc-Tyr(tBu)-Aib-Glu(OtBu)-Gly-Thr(tBu)-Phe-Thr(tBu)-Ser(tBu)-Asp(OtBu)-Tyr(tBu)-Ser(tBu)-Ile-Aib-Leu-Asp(OtBu)-Lys(Boc)-Ile-Ala-Gln(Trt)-Lys(NH-Compound I-di-tertbutyl ester)-Ala-Phe-Val-Gln(Trt)-Trp-Leu-Ile-Ala-Gly-Gly-Pro-Ser(tBu)-Ser(tBu)-Gly-Ala-Pro-Pro-Pro-Ser(tBu)-resin.

De-blocking of the Fmoc-Tyr(tBu)-Aib-Glu(OtBu)-Gly-Thr(tBu)-Phe-Thr(tBu)-Ser(tBu)-Asp(OtBu)-Tyr(tBu)-Ser(tBu)-Ile-Aib-Leu-Asp(OtBu)-Lys(Boc)-Ile-Ala-Gln(Trt)-Lys(NH-Compound I-di-tert-butyl ester)-Ala-Phe-Val-Gln(Trt)-Trp-Leu-Ile-Ala-Gly-Gly-Pro-Ser(tBu)-Ser(tBu)-Gly-Ala-Pro-Pro-Pro-Ser(tBu)-resin was carried out using piperidine followed by Boc protection of the peptide resin using Boc anhydride to yield the intermediate compound, Boc-Tyr(tBu)-Aib-Glu(OtBu)-Gly-Thr(tBu)-Phe-Thr(tBu)-Ser(tBu)-Asp(OtBu)-Tyr(tBu)-Ser(tBu)-Ile-Aib-Leu-Asp(OtBu)-Lys(Boc)-Ile-Ala-Gln(Trt)-Lys(NH-Compound I-di-tert-butyl ester)-Ala-Phe-Val-Gln(Trt)-Trp-Leu-Ile-Ala-Gly-Gly-Pro-Ser(tBu)-Ser(tBu)-Gly-Ala-Pro-Pro-Pro-Ser(tBu)-resin, which on cleavage and de-protection using trifluoroacetic acid with a scavenger gave the crude product, which was further purified through preparative HPLC to provide tirzepatide.

### Example 5: Preparation of Tirzepatide—Method D

The parent peptide is synthesized by solid-phase method using fragment coupling to increase the purity. Different fragments are synthesised using 2-chlorotrityl chloride resin and cleaved from the resin using trifluoroethanol: DCM (1:1) or 0.1% TFA in DCM or dilute hydrochloride solution in DCM to yield protected fragments. Protected fragments of peptide are coupled by SPPS, LPPS or hybrid SPPS/LPPS approaches using Coupling reagent/Auxiliary nucleophile/Base.

In addition, it should be understood that the scope of the present disclosure is not limited to the above-described examples, and those skilled in the art will appreciate that various modifications and alterations are possible without departing from the scope of the present disclosure. For example, the batch sizes may be altered by a person having ordinary skill in the art while staying within the present disclosure.

### Example 6: Preparation of SEQ ID NO: 1 to SEQ ID NO. 10

### Solid Phase Peptide Synthesis—General Procedure-Attachment of First Amino Acid on Solid Support.

### 1. 2-Chlorotrityl Chloride Resin—

Charged 2-Chlorotrityl chloride resin to solid phase peptide synthesis glass assembly.
Washed resin with DCM (Dichloromethane).
Coupled Fmoc-AA-OH using DIPEA as base and allowed reaction mixture to stir.
Washed resin with DMF/THF.

### 2. Fmoc-Rink Amide AM Resin—

Charged Fmoc-Rink Amide AM resin to solid phase peptide synthesis glass assembly.
Washed resin with DMF.
Fmoc-deprotected using a piperidine/DMF solution at room temperature.
Washed the resin to remove piperidine by washoffing with DMF, IPA and THF. Checked for neutral pH using pH paper.
Checked in-process test and proceeded further.
Coupled Fmoc-AA-OH using DIPC/HOBt and allowed the reaction mixture to stir until completion of reaction
Checked in-process test and proceeded further.
Washed resin with solvent.

### 3. Fmoc-Removal (Deblocking)—

Washed resin with DMF
Fmoc-removed using a 50% piperidine/DMF solution at room temperature.
Washed resin to remove piperidine with DMF, IPA and THF. Checked pH using pH paper. pH must be 6-7. If not repeated DMF wash to achieve the pH 6-7 using pH paper.
Checked in-process test and proceeded further.

### 4. Coupling—

Washed resin with THF:DMF.
Coupled Fmoc-AA-OH using DIPC/HOBt.
Allowed reaction mixture to stir.
Checked in-process test and proceeded further.
If process completed, proceeded to washing and if not complete, repeated coupling.
Washed resin with solvent.
   A. Preparation of Protected SEQ ID NO: 1 Fmoc-Thr(tBu)⁵-Phe-Thr(tBu)-Ser(tBu)⁸:

The solid phase synthesis of the fragment was done using 2-Cl trityl resin with a loading of 1 to 1.2 mmol/g. After coupling of Fmoc-Ser(tBu)-OH loading observed was 1.2 mm/g. The general solid phase peptide synthesis was followed including coupling, and deblocking. Details of equivalent of protected amino acid, reagent and solvent were as below:

| S. No. | Amino Acid | Condition |
|---|---|---|
| 1 | Fmoc-Ser(tBu)-OH | Solvent-Dichloromethane |
| | | 2.0 eq AA/2.5 eq base, 2 h, Room temperature |
| 2 | Fmoc-Thr(tBu)-OH | Deblocking- |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 2 Hrs RT |
| 3 | Fmoc-Phe-OH | Deblocking |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 2 Hrs RT |
| 4 | Fmoc-Thr(tBu)-OH | Deblocking |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 2 Hrs RT |

Protected peptide fragment Fmoc-Thr(tBu)-Ser(tBu)-Phe-Thr(tBu)-OH was cleaved and isolated from the solid support using trifluoroethanol: DCM (1:1)-10V of starting resin. Washed the resin with dichloromethane. Prepared a solution of 2,2,2-trifluoroethanol (TFE)/dichloromethane (DCM). Charged this solution to the resin and stirred the mixture for 60 minutes at room temperature. The reaction mixture was dried with suction and then the filtrate was collected. The solvent was distilled out of the filtrate on a rotavapour at 35° C.-40° C. The above cycle was repeated two times, to provide a thick syrup. n-Hexane was charged to the thick syrup, and the resulting mixture was co-distilled out on a rotavapour at 35° C.-40° C. to provide a solid. n-Hexane was charged to the above solid. The resulting mixture was stirred and filtered. Dried the resulting solid on a rotavapour at 35° C.-40° C. Dried the resulting solid powder on rotavapor. 16 Gram (>90% Yield) 99% Purity

B: SEQ ID NO. 2-Fmoc-Asp(OtBu)⁹-Tyr(tBu)-Ser(tBu)-Ile-Aib-Leu¹⁴ (9-14)

The solid phase synthesis of fragment was done using 30 g 2-Cl trityl resin with a loading of 1 to 1.2 mmol/g. After coupling of Fmoc-Leu-OH loading observed was 1.0 mm/g. The general solid phase peptide synthesis was followed including coupling, deblocking. Details of equivalent of protected amino acid, reagent and solvent were as below

| S. No. | Amino Acid | Condition |
|---|---|---|
| 1 | Fmoc-Leu-OH | Solvent-Dichloromethane |
| | | 2.0 eq AA/2.5 eq base, 2 h, Room temperature |
| 2 | Fmoc-Aib-OH | Deblocking- |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 2 Hrs RT |
| 3 | Fmoc-Ile-OH | Deblocking |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 2 Hrs RT |
| 4 | Fmoc-Ser(tBu)-OH | Deblocking |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 2 Hrs RT |
| 5 | Fmoc-Tyr(tBu)-OH | Deblocking |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 2 Hrs RT |
| 6 | Fmoc-Asp(OtBu)-OH | Deblocking |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 2 Hrs RT |

Cleavage from Solid support—Protected peptide fragment Fmoc-Asp(OtBu)-Tyr(tBu)-Ser(tBu)-Ile-Aib-Leu-OH was isolated from solid support using Trifluoroethanol: DCM (1:1)-10 V of starting resin. Procedure for cleavage followed was as mentioned for SEQ ID NO: 1.

Dried the solid powder on a rotavapor. 27 Gram (9000 Yield) 980% Purity

C. SEQ ID NO. 3—Fmoc-Ala¹⁸-Gln(Trt)-Lys(Side Chain)*-Ala-Phe-Val-Gln(Trt)-Trp²⁵-Leu-Ile-Ala-Gly²⁹-OH

The solid phase synthesis of fragment was done using 10 g 2-Cl trityl resin with a loading of 1 to 1.2 mmol/g. After coupling of Fmoc-Gly-OH loading observed was 0.8 mm/g. The general solid phase peptide synthesis was followed including coupling, and deblocking. Details of equivalent of protected amino acid, reagent and solvent were as below

| S. No. | Amino Acid | Condition |
|---|---|---|
| 1 | Fmoc-Gly-OH | Solvent-Dichloromethane |
| | | 2.0 eq AA/2.5 eq base, 2 h, Room temperature |
| 2 | Fmoc-Ala-OH | Deblocking- |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 2 Hrs RT |
| 3 | Fmoc-Ile-OH | Deblocking |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 2 Hrs RT |
| 4 | Fmoc-Leu-OH | Deblocking |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 2 Hrs RT |
| 5 | Fmoc-Trp-OH | Deblocking |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 2 Hrs RT |
| 6 | Fmoc-Gln(Trt)-OH | Deblocking |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 2 Hrs RT |
| 7 | Fmoc-Val-OH | Deblocking |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 2 Hrs RT |
| 8 | Fmoc-Phe-OH | Deblocking |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 2 Hrs RT |
| 9 | Fmoc-Ala-OH | Deblocking |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 2 Hrs RT |
| 10 | Fmoc-Lys(Side chain)-OH | Deblocking |
| | | 50% Piperidine/DMF 30 min RT |
| | | Check pH |
| | | Coupling- |
| | | Solvent-Tetrahydrofuran/DMF |
| | | 1.5 eq AA/1.5 eq HOBt/1.5 eq DIPC, 24 Hrs RT |
| 11 | Fmoc-Gln(Trt)-OH | Deblocking |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 2 Hrs RT |
| 12 | Fmoc-Ala-OH | Deblocking |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 2 Hrs RT |

Cleavage from Solid support—Protected peptide fragment Fmoc-Ala-Gln(Trt)-Lys(Side Chain)*-Ala-Phe-Val-Gln(Trt)-Trp25-Leu-Ile-Ala-Gly-OH was isolated from solid support using Trifluoroethanol: DCM (1:1)-10V of starting resin. Procedure for cleavage followed was as mentioned for SEQ ID NO: 1.

Dried the solid powder on a rotavapor. 24 Gram (8300 Yield) 92% Purity

D. SEQ ID NO. 3 (with Protecting Group)-Fmoc-Ala¹⁸-Gln(Trt)-Lys(ivdde)*-Ala Phe-Val-Gln(Trt)-Trp²⁵-Leu-Ile-Ala-Gly²⁹-OH

The solid phase synthesis of fragment was done using 15 g 2-Cl trityl resin with a loading of 1 to 1.2 mmol/g. After coupling of Fmoc-Gly-OH loading observed was 1.0 mm/g. The general solid phase peptide synthesis was followed including coupling, and deblocking. Details of equivalent of protected amino acid, reagent and solvent were as below:

| S. No. | Amino Acid | Condition |
|---|---|---|
| 1 | Fmoc-Gly-OH | Solvent-Dichloromethane |
| | | 2.0 eq AA/2.5 eq base, 2 h, Room temperature |
| 2 | Fmoc-Ala-OH | Deblocking- |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 2 Hrs RT |
| 3 | Fmoc-Ile-OH | Deblocking |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 2 Hrs RT |
| 4 | Fmoc-Leu-OH | Deblocking |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 2 Hrs RT |
| 5 | Fmoc-Trp-OH | Deblocking |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 2 Hrs RT |
| 6 | Fmoc-Gln(Trt)-OH | Deblocking |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 2 Hrs RT |
| 7 | Fmoc-Val-OH | Deblocking |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 2 Hrs RT |
| 8 | Fmoc-Phe-OH | Deblocking |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 2 Hrs RT |
| 9 | Fmoc-Ala-OH | Deblocking |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 2 Hrs RT |
| 10 | Fmoc-Lys(iVdde)-OH | Deblocking |
| | | 50% Piperidine/DMF 30 min RT |
| | | Check pH |
| | | Coupling- |
| | | Solvent-Tetrahydrofuran/DMF |
| | | 2 eq AA/2 eq HOBt/1.5 eq DIPC, 24 Hrs RT |
| 11 | Fmoc-Gln(Trt)-OH | Deblocking |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 2 Hrs RT |
| 12 | Fmoc-Ala-OH | Deblocking |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 2 Hrs RT |

Cleavage from Solid support—Protected peptide fragment Fmoc-Ala-Gln(Trt)-Lys(iVdde)*-Ala-Phe-Val-Gln(Trt)-Trp²⁵-Leu-Ile-Ala-Gly-OH was isolated from solid support using Trifluoroethanol: DCM (1:1)-10 V of starting resin. Procedure for cleavage followed was as mentioned for SEQ ID NO: 1.

Dried the solid powder on a rotavapor. 32 Gram (95% Yield) 95% Purity.

### E. SEQ ID NO. 4-Fmoc-Lys(Side Chain)-Ala²¹-OH

The solid phase synthesis of fragment was done using 10 g 2-Cl trityl resin with a loading of 1 to 1.2 mmol/g. After coupling of Fmoc-Ala-OH loading observed was 1.0 mm/g. The general solid phase peptide synthesis was followed including coupling, and deblocking. Details of equivalent of protected amino acid, reagent and solvent were as below:

| S. No. | Amino Acid | Condition |
|---|---|---|
| 1 | Fmoc-Ala-OH | Solvent-Dichloromethane |
| | | 2.0 eq AA/2.5 eq base, 2 h, Room temperature |
| 2 | Fmoc-Lys(Side | Deblocking- |
| | chain)-OH | 50% Piperidine/DMF 30 min RT |
| | | Check pH |
| | | Coupling- |
| | | Solvent-Tetrahydrofuran/DMF |
| | | 1.5 eq AA/1.5 eq HOBt/1.5 eq DIPC, 4 Hrs RT |

Cleavage from Solid support—Protected peptide fragment Fmoc-Lys(Side Chain)*-Ala-OH support using Trifluoroethanol: DCM (1:1)-10V of starting resin. Procedure for cleavage followed was as mentioned for SEQ ID NO: 1.

Dried the Sticky material on a rotavapor. 11.4 Gram (88% Yield) 92% Purity

F. SEQ ID NO. 5-Boc-Tyr(tBu)¹-Aib-Glu(OtBu)-Gly-Thr(tBu)-Phe-Thr(tBu) Ser(tBu)⁸-OH

The solid phase synthesis of fragment was done using 30 g 2-Cl trityl resin with a loading of 1 to 1.2 mmol/g. After coupling of Fmoc-(5-8)-OH loading observed was 1 mm/g. The general solid phase peptide synthesis was followed including coupling, and deblocking. Details of equivalent of protected amino acid, reagent and solvent were as below:

| S. No. | Amino Acid | Condition |
|---|---|---|
| 1 | Fmoc-(5-8)-OH | Solvent-Dichloromethane |
| | Fmoc-Thr(tBu)- | 1.5 eq AA/2 eq base, 2 h, Room temperature |
| | Phe-Thr(tBu)- | |
| | Ser(tBu)-OH | |
| 2 | Fmoc-Gly-OH | Deblocking- |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 2 Hrs RT |
| 3 | Fmoc-Glu(OtBu)- | Deblocking |
| | OH | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 2 Hrs RT |
| 4 | Fmoc-Aib-OH | Deblocking |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 3 Hrs RT |
| 5 | Boc-Tyr(tBu)-OH | Deblocking |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 4 Hrs RT |

Cleavage from Solid support—Protected peptide fragment Boc-Tyr(tBlu)1-Aib-Glu(OtBu)-Gly-Thr(tBu)-Phe-Thr(tBu)-Ser(tBlu)-OH was cleaved from the support using trifluoroethanol: DCM (1:1)-10 V of starting resin. Procedure for cleavage followed was as mentioned for SEQ ID NO: 1.

Dried the solid powder on a rotavapor. 34 Gram (9300 Yield) 9600 Purity

G. SEQ ID NO. 6-Boc-Tyr(tBu)¹-Aib-Glu(OtBu)-Gly-Thr(tBu)-Phe-Thr(tBu) Ser(tBu)-Asp(OtBu)-Tyr(tBu)-Ser(tBu)¹¹-OH

The solid phase synthesis of fragment was done using bag 2-Cl trityl resin with a loading of 1 to 1.4 mmol/g. After coupling of Fmoc-Ser(tBu)-OH loading observed was 1.3 mm/g. The general solid phase peptide synthesis was followed including coupling, and deblocking. Details of equivalent of protected amino acid, reagent and solvent were as below:

| S. No. | Amino Acid | Condition |
|---|---|---|
| 1 | Fmoc-Ser(tBu)-OH | Solvent-Dichloromethane |
| | | 2.0 eq AA/2.5 eq base, 2 h, Room temperature |
| 2 | Fmoc-Tyr(tBu)-OH | Deblocking- |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 2 Hrs RT |
| 3 | Fmoc-Asp(OtBu)-OH | Deblocking |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 2 Hrs RT |
| 4 | Fmoc-Ser(tBu)-OH | Deblocking |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 2 Hrs RT |
| 5 | Fmoc-Thr(tBu)-OH | Deblocking- |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 2 Hrs RT |
| 6 | Fmoc-Phe-OH | Deblocking |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 2 Hrs RT |
| 7 | Fmoc-Thr(tBu)-OH | Deblocking |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 2 Hrs RT |
| 8 | Fmoc-Gly-OH | Deblocking- |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 2 Hrs RT |
| 9 | Fmoc-Glu(OtBu)-OH | Deblocking |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 2 Hrs RT |
| 10 | Fmoc-Aib-OH | Deblocking |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 3 Hrs RT |
| 11 | Boc-Tyr(tBu)-OH | Deblocking |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 4 Hrs RT |

Cleavage from Solid support—Protected peptide fragment Boc-Tyr(tBlu)¹-Aib-Glu(OtBu)-Gly-Thr(tBu)-Phe-Thr(tBu)-Ser(tBlu)-Asp(OtBu)-Tyr(tBu)-Ser(tBlu)¹¹-OH was cleaved from the support using trifluoroethanol: DCM (1:1)-10 V of starting resin. Procedure for cleavage followed was as mentioned for SEQ ID NO 1.

Dried the solid powder on a rotavapor. 230 Gram (9700 Yield) 8600 Purity

H. SEQ ID NO. 10D-Fmoc-Ile¹²-Aib-Len-Asp(OtBu)-Lys(Boc)-Ile-Ala-Gln(Trt) Lys(Side Chain)-Ala²¹-Phe-Val-Gln(Trt)-Trp²⁵-Leu-Ile-Ala-Gly²⁹-OH

The solid phase synthesis of fragment was done using 10 g 2-Cl trityl resin with a loading of 1 to 1.4 mmol/g. After coupling of Fmoc-Gly-OH loading observed was 1.0 mm/g. The general solid phase peptide synthesis was followed including coupling, and deblocking. Details of equivalent of protected amino acid, reagent and solvent were as below:

| S. No. | Amino Acid | Condition |
|---|---|---|
| 1 | Fmoc-Gly-OH | Solvent-Dichloromethane |
| | | 2.0 eq AA/2.5 eq base, 2 h, Room temperature |
| 2 | Fmoc-Ala-OH | Deblocking- |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 2 Hrs RT |
| 3 | Fmoc-Ile-OH | Deblocking |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 2 Hrs RT |
| 4 | Fmoc-Leu-OH | Deblocking |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 2 Hrs RT |
| 5 | Fmoc-Trp-OH | Deblocking |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 2 Hrs RT |
| 6 | Fmoc-Gln(Trt)-OH | Deblocking |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 2 Hrs RT |
| 7 | Fmoc-Val-OH | Deblocking |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 2 Hrs RT |
| 8 | Fmoc-Phe-OH | Deblocking |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 2 Hrs RT |
| 9 | Fmoc-Ala-OH | Deblocking |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 2 Hrs RT |
| 10 | Fmoc-Lys(Side chain)- | Deblocking |
| | OH | 50% Piperidine/DMF 30 min RT |
| | | Check pH |
| | | Coupling- |
| | | Solvent-Tetrahydrofuran/DMF |
| | | 1.5 eq AA/1.5 eq HOBt/1.5 eq DIPC, 24 Hrs RT |
| 11 | Fmoc-Gln(Trt)-OH | Deblocking |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 2 Hrs RT |
| 12 | Fmoc-Ala-OH | Deblocking |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 2 Hrs RT |
| 13 | Fmoc-Ile-OH | Deblocking |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 6 Hrs RT |
| 14 | Fmoc-Lys(Boc)-OH | Deblocking |
| | | 50% Piperidine/DMF 30 min RT |
| | | Check pH |
| | | Coupling- |
| | | Solvent-Tetrahydrofuran/DMF |
| | | 1.5 eq AA/1.5 eq HOBt/1.5 eq DIPC, 2 Hrs RT |
| 15 | Fmoc-Asp(OtBu)-OH | Deblocking |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 2 Hrs RT |
| 16 | Fmoc-Leu-OH | Deblocking |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 2 Hrs RT |
| 17 | Fmoc-Aib-OH | Deblocking |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 3 Hrs RT |
| 18 | Fmoc-Ile-OH | Deblocking |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 4 Hrs RT |

Cleavage from Solid support—Protected peptide fragment Fmoc-Ile-Aib-Leu-Asp(OtBu)-Lys(Boc)-Ile-Ala-Gln(Trt)-Lys(Side Chain)-Ala²¹-Phe-Val-Gln(Trt)-Trp²⁵-Leu-Ile-Ala-Gly-OH was cleaved from the support using trifluoroethanol: DCM (1:1)-10 V of starting resin. Procedure for cleavage followed was as mentioned for SEQ ID NO: 1.

Dried the solid powder on a rotavapor. 30 Gram (8000 Yield) 700% Purity.

I. SEQ ID NO. 8-Fmoc-Lys(Boc)¹⁶-Ile-Ala-Gln(Trt)-Lys(Side chain)*-Ala-Phe-Val-Gln(Trt)-Trp-Leu-Ile-Ala -Gly²⁹-OH

The solid phase synthesis of fragment was done using 2-Cl trityl resin with a loading of 1 to 1.4 mmol/g. After coupling of Fmoc-Gly-OH loading observed was 1.0 mm/g. The general solid phase peptide synthesis was followed including coupling, and deblocking. Details of equivalent of protected amino acid, reagent and solvent were as below:

| S. No. | Amino Acid | Condition |
|---|---|---|
| 1 | Fmoc-Gly-OH | Solvent-Dichloromethane |
| | | 2.0 eq AA/2.5 eq base, 2 h, Room temperature |
| 2 | Fmoc-Ala-OH | Deblocking- |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 2 Hrs RT |
| 3 | Fmoc-Ile-OH | Deblocking |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 2 Hrs RT |
| 4 | Fmoc-Leu-OH | Deblocking |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 2 Hrs RT |
| 5 | Fmoc-Trp-OH | Deblocking |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 2 Hrs RT |
| 6 | Fmoc-Gln(Trt)-OH | Deblocking |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 2 Hrs RT |
| 7 | Fmoc-Val-OH | Deblocking |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 2 Hrs RT |
| 8 | Fmoc-Phe-OH | Deblocking |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 2 Hrs RT |
| 9 | Fmoc-Ala-OH | Deblocking |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 2 Hrs RT |
| 10 | Fmoc-Lys(Side chain)- | Deblocking |
| | OH | 50% Piperidine/DMF 30 min RT |
| | | Check pH |
| | | Coupling- |
| | | Solvent-Tetrahydrofuran/DMF |
| | | 1.5 eq AA/1.5 eq HOBt/1.5 eq DIPC, 24 Hrs RT |
| 11 | Fmoc-Gln(Trt)-OH | Deblocking |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 2 Hrs RT |
| 12 | Fmoc-Ala-OH | Deblocking |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 2 Hrs RT |
| 13 | Fmoc-Ile-OH | Deblocking |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 6 Hrs RT |
| 14 | Fmoc-Lys(Boc)-OH | Deblocking |
| | | 50% Piperidine/DMF 30 min RT |
| | | Check pH |
| | | Coupling- |
| | | Solvent-Tetrahydrofuran/DMF |
| | | 1.5 eq AA/1.5 eq HOBt/1.5 eq DIPC, 2 Hrs RT |

Cleavage from Solid support—Protected peptide fragment Fmoc-Lys(Boc)-Ile-Ala-Gln(Trt)-Lys(Side Chain)-Ala21-Phe-Val-Gln(Trt)-Trp25-Leu-Ile-Ala-Gly-OH was cleaved from the support using trifluoroethanol: DCM (1:1)-10V of starting resin. Procedure for cleavage followed was as mentioned for SEQ ID NO 1.

Dried the solid powder on a rotavapor. 38 Gram (9800 Yield) 80% Purity

J. SEQ ID NO. 9-Boc-Tyr(tBu)^{t}-Aib-Glu(OtBu)-Gly⁴-OH

The solid phase synthesis of fragment was done using 100 g 2-Cl trityl resin with a loading of 1 to 1.4 mmol/g. After coupling of Fmoc-Gly-OH loading observed was 1.1 mm/g. The general solid phase peptide synthesis was followed including coupling, and deblocking. Details of equivalent of protected amino acid, reagent and solvent were as below:

| S. No. | Amino Acid | Condition |
|---|---|---|
| 1 | Fmoc-Gly-OH | Solvent-Dichloromethane |
| | | 2 eq AA/2.5 eq base, 2 h, Room temperature |
| 2 | Fmoc-Glu(OtBu)-OH | Deblocking- |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 2 Hrs RT |
| 3 | Fmoc-Aib-OH | Deblocking |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 2 Hrs RT |
| 4 | Boc-Tyr(tBu)-OH | Deblocking |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 3 Hrs RT |

Cleavage from Solid support—Protected peptide fragment Fmoc-Lys(Boc)-Ile-Ala-Gln(Trt)-Lys(Side Chain)-Ala²¹-Phe-Val-Gln(Trt)-Trp²⁵-Leu-Ile-Ala-Gly-OH was cleaved from the solid support using trifluoroethanol: DCM (1:1)-10 volume of starting resin. Procedure for cleavage followed was as mentioned for SEQ ID NO: 1.

Dried the solid powder on a rotavapor. 67 Gram (91% Yield) 96% Purity

### General Processes for Tirzepatide Synthesis Using SEQ ID NO: 1 to SEQ ID NO: 10:

### 1. Fmoc-Rink Amide AM Resin—

Charged Fmoc-Rink Amide AM resin to solid phase peptide synthesis glass assembly.
Washed resin with DMF.
Fmoc-deprotected using piperidine/DMF solution at room temperature.
Washed resin to remove piperidine by wash of DMF, IPA and THF. Checked neutral pH using pH paper.
Checked in-process test and proceeded further.
Coupled Fmoc-AA-OH using DIPC/HOBt and allowed the reaction mixture to stir until completion of reaction.
Checked in-process test and proceeded further.
Washed resin with solvent.

### 2. Fmoc-Removal (Deblocking)—

Washed resin with DMF.
Fmoc-removed using 50% piperidine/DMF solution at room temperature.
Washed resin to remove piperidine by wash of DMF, IPA and THF. Checked pH using pH paper. pH must be 6-7. If not, repeated DMF wash to achieve the pH 6-7 using pH paper.
Checked in-process test and proceeded further.

### 3. Coupling—

Washed resin with THF:DMF.
Coupled Fmoc-AA-OH using DIPC/HOBt.
Allowed reaction mixture to stir.
Checked in-process test and proceeded further.
If passes proceeded for washing and if not-passes, proceeded for repeat coupling.
Washed resin with solvent.

### Example 7: Preparation of Fmoc-Gly³⁰-Pro-Ser(tBu)-Ser(tBu)-Gly-Ala-Pro-Pro-Pro-Ser³⁹-Resin (Fmoc-30-39-RESIN)

Charged 50 g Fmoc-Rink amide AM resin (Substitution 0.5 mm/g) to SPPS assembly and washed with DMF. Charged piperidine/DMF solution to resin and stirred for 30 min and washed with solvent to get pH neutral. Checked in-process test for confirmation of Fmoc-removal from the resin.

Dissolved Fmoc-Ser(tBu)-OH (19.2 g, 50 mm, 2 eq) and HOBt (7.7 g, 50 mm, 2 eq) in THF:DMF. To above solution, added DIPC (6.4 g, 50 mm, 2 eq) and added this reaction mixture to resin and allowed the resulting mixture to stir at 20 to 30 C. Checked in-process test to check the completion of the reaction and after completion of the reaction suction dried the reaction mixture and washed the resin with DMF/IPA/THF. Capped the unreacted reaction site using acetic anhydride.

This is Fmoc-Ser(tBu)-Rink Amide AM resin.

Next amino acid was coupled as per sequence using solid phase peptide synthesis including deblocking, coupling, and capping. Details of equivalent of protected amino acid, reagent and solvent were as below:

| S. No. | Amino Acid | Condition |
|---|---|---|
| 1 | Fmoc-Pro-OH | Deblocking- |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 3 Hrs RT |
| 2 | Fmoc-Pro-OH | Deblocking |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 3 Hrs RT |
| 3 | Fmoc-Pro-OH | Deblocking- |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 3 Hrs RT |
| 4 | Fmoc-Ala-OH | Deblocking- |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 3 Hrs RT |
| 5 | Fmoc-Gly-OH | Deblocking |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 3 Hrs RT |
| 6 | Fmoc-Ser(tBu)-OH | Deblocking |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 3 Hrs RT |
| 7 | Fmoc-Ser(tBu)-OH | Deblocking- |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 3 Hrs RT |
| 8 | Fmoc-Pro-OH | Deblocking |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 3 Hrs RT |
| 10 | Fmoc-Gly-OH | Deblocking |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 3 Hrs RT |

This is Fmoc-Gly30-Pro-Ser(tu)-Ser(tBu)-Gly-Ala-Pro-Pro-Pro-Ser39-Resin (Fmoc-30-39-RESIN), which was used for the fragment coupling to synthesise tirzepatide.

### Example 8: Preparation of Tirzepatide

Fmoc-deblocking of 15 g/5 mm Fmoc-(30-39)-Resin (Example 7) used piperidine/DMF and stirred for 30 minute and then washed with solvent to get pH neutral. Checked in-process test for confirmation of Fmoc-removal from the resin. This is H-Gly³⁰-Pro-Ser(tu)-Ser(tBu)-Gly-Ala-Pro-Pro-Pro-Ser³⁹-Resin.

Next amino acid/Fragment was coupled as per sequence using solid phase peptide synthesis including deblocking, coupling, and capping. Details of equivalent of protected amino acid/fragment, reagent and solvent were as below.

| S. No. | Amino Acid | Condition |
|---|---|---|
| 1 | Fmoc-Ala18-Gln(Trt)- | Coupling- |
| | Lys20(Side chain)-Ala-Phe- | Solvent-DMF |
| | Val-Gln(Trt)-Trp25- | 1.6 eq AA/1.6 eq PyBOP/1.6 eq HOBt and |
| | Leu-Ile-Ala-Gly29-OH | DIPEA, 24 Hrs RT |
| 2 | Fmoc-Ile-OH | Deblocking |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 6 Hrs RT |
| 3 | Fmoc-Lys(Boc)-OH | Deblocking- |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 3 Hrs RT |
| 4 | Fmoc-Asp(OtBu)-OH | Deblocking- |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 3 Hrs RT |
| 5 | Fmoc-Asp(OtBu)9- | Deblocking |
| | Tyr(tBu)-Ser(tBu)-Ile- | 50% Piperidine/DMF 30 min RT |
| | Aib-Leu-OH | Check pH |
| | | Coupling- |
| | | Solvent-Tetrahydrofuran/DMF |
| | | 2.0 eq AA/2 eq HOBt/2.0 eq DIPC, 4 Hrs RT |
| 6 | Fmoc-Thr(tBu)5-Phe- | Deblocking |
| | Thr(tBu)-Ser(tBu)8-OH | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 3 Hrs RT |
| 7 | Boc-Tyr(tBu)1-Aib- | Deblocking- |
| | Glu(OtBu)-Gly-OH | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 3 Hrs RT |

On completion of solid phase synthesis, the resin was removed from assembly and dried to get Boc-Tyr(tBu)¹-Aib-Glu(OtBu)-Gly-Thr(tBu)⁵-Phe-Thr(tBu)-Ser(tBu)-Asp(OtBu)-Tyr(tBu)¹⁰-Ser(tBu)-Ile-Aib-Leu-Asp(OtBu)¹⁵-Lys(Boc)-Ile-Ala-Gln(Trt)-Lys²⁰(Side chain)-Ala-Phe-Val-Gln(Trt)-Trp²⁵-Leu-Ile-Ala-Gly-Gly³⁰-Pro-Ser(tBu)-Ser(tBu)-Gly-Ala³⁵-Pro-Pro-Pro-Ser(tBu)³⁹-Rink Resin-23 g (Boc-(01-39)-Rink Resin).

20 g of Boc-(01-39)-Rink Resin was washed with dichloromethane and suspended in 200 mL TFA/ethanedithiol (95:5) for 1 hour at temperature 20° C. to 30° C. The reaction mixture was suction dried and the filtrate was collected. The resin was further was with 2×25 mL TFA and then mixed with the filtrate. Distilled out the filtrate on a rotavapor at 20° C.-30° C. to get a sticky syrup. The sticky syrup was triturated with 100 mL diethylether and further washed with diethyl ether to get a crude solid −10 g.

### Purification of Tirzepatide

The tirzepatide peptide crude 10 g obtained above was dissolved in a buffer of pH 8.5 and acetonitrile. The tirzepatide crude peptide solution was subjected to purification on octadecyl bonded silica gel with gradient of a phosphate buffer of pH 8 and acetonitrile. The tirzepatide enriched fraction was again purified with TFA in water and acetonitrile. The impure pool was further purified with a phosphate buffer of pH 2.5 and acetonitrile. The pure fraction of tirzepatide was desalted as a sodium salt, and the acetonitrile was removed from eluent by distillation over a rotavapor. The concentrated solution was taken for freeze drying to obtain 1.1 g tirzepatide with 98.8200 HPLC purity.

Tirzepatide Average Mass-4813.4 Observed Mass—1205 (M+4H)⁺⁴

### Example-9 Preparation of Tirzepatide

Fmoc-deblocking of 15 g/5 mm Fmoc-(30-39)-Resin (Example 7) was done using piperidine/DMF. The mixture was stirred for 30 minute and then washed with a solvent to get to a neutral pH. Checked in-process test for confirmation of Fmoc-removal from the resin. This was H-Gly30-Pro-Ser(tBu)-Ser(tBlu)-Gly-Ala-Pro-Pro-Pro-Ser39-Resin (H-30-39-RESIN),

Next amino acid/Fragment was coupled as per sequence using solid phase peptide synthesis including deblocking, coupling, and capping. Details of equivalent of protected amino acid/fragment, reagent and solvent were as below:

| S. No. | Amino Acid | Condition |
|---|---|---|
| 1 | Fmoc-Ala18-Gln(Trt)- | Coupling- |
| | Lys20(ivdde)-Ala-Phe- | Solvent-DMF |
| | Val-Gln(Trt)-Trp25-Leu- | 1.6 eq AA/1.6 eq PyBOP/1.6 eq HOBt and DIPEA, |
| | Ile-Ala-Gly29-OH | 24 Hrs RT |
| 2 | Fmoc-Ile-OH | Deblocking |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 6 Hrs RT |
| 3 | Fmoc-Lys(Boc)-OH | Deblocking- |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 3 Hrs RT |
| 4 | Fmoc-Asp(OtBu)-OH | Deblocking- |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 3 Hrs RT |
| 5 | Fmoc-Asp(OtBu)9- | Deblocking |
| | Tyr(tBu)-Ser(tBu)-Ile- | 50% Piperidine/DMF 30 min RT |
| | Aib-Leu-OH | Check pH |
| | | Coupling- |
| | | Solvent-Tetrahydrofuran/DMF |
| | | 2.0 eq AA/2 eq HOBt/2.0 eq DIPC, 4 Hrs RT |
| 6 | Fmoc-Thr(tBu)5-Phe- | Deblocking |
| | Thr(tBu)-Ser(tBu)8-OH | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 3 Hrs RT |
| 7 | Boc-Tyr(tBu)1-Aib- | Deblocking- |
| | Glu(OtBu)-Gly-OH | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 3 Hrs RT |
| 8 | Deprotection of ivdde | Washed resin with DMF |
| | | Charged 5% Hydrazine/DMF solution to resin |
| | | and stirred for 1 Hrs. |
| | | Washed resin with DMF/TPA/THF |
| 9 | Coupling of Fmoc-AEEA- | Coupling- |
| | OH | Solvent-Tetrahydrofuran/DMF |
| | | 2.0 eq AA/2 eq HOBt/2.0 eq DIPC, 3 Hrs RT |
| 10 | Coupling of Fmoc-AEEA- | Deblocking- |
| | OH | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 3 Hrs RT |
| 11 | Coupling of Fmoc-GLU- | Deblocking- |
| | OTBU | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 4 Hrs RT |
| 12 | Coupling of Ecosanedioic | Deblocking- |
| | acid mono tert-butyl ester | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 4 Hrs RT |

On completion of solid phase synthesis the resin was removed from the assembly and dried to get Boc-Tyr(tBu)¹⁰-Aib-Glu(OtBu)-Gly-Thr(tBu)⁵-Phe-Thr(tBu)-Ser(tBu)-Asp(OtBu)-Tyr(tBu)¹⁰-Ser(tBu)-Ile-Aib-Leu-Asp(OtBu)¹⁵-Lys(Boc)-Ile-Ala-Gln(Trt)-Lys²⁰(Side chain)-Ala-Phe-Val-Gln(Trt)-Trp²⁵-Leu-Ile-Ala-Gly-Gly³⁰-Pro-Ser(tBu)-Ser(tBu)-Gly-Ala35-Pro-Pro-Pro-Ser(tBu)³⁹-Rink Resin-32 g (Boc-(01-39)-Rink Resin).

30 g of Boc-(01-39)-Rink Resin was washed with dichloromethane and suspended in 200 mL TFA/ethanedithiol (95:5) for 1 Hour at a temperature of 20 to 30° C. The reaction mixture was suction dried, and the filtrate was collected. The resin was further washed with 2×25 mL TFA and then mixed with the filtrate.

Distilled out filtrate on a rotavapor at 20-30 C to get a sticky syrup. Triturated the sticky syrup with 100 mL diethylether and further washed the mixture with diethyl ether to get a crude solid −15 g.

### Purification of Tirzepatide

The tirzepatide peptide crude 10 g obtained above was dissolved in a buffer of pH 8.5 and acetonitrile. The tirzepatide crude peptide solution was subjected to purification on octadecyl bonded silica gel with a gradient of phosphate buffer of pH 8 and acetonitrile. The tirzepatide enriched fraction was again purified with TFA in water and acetonitrile, and the impure pool was further purified with a phosphate buffer of pH 2.5 and acetonitrile. The pure fraction of tirzepatide was desalted as a sodium salt, and the acetonitrile was removed from the eluent by distillation over a rotavapor. The concentrated solution was taken for freeze drying to obtain 1.3 g tirzepatide with 99.43% PLC purity.

Tirzepatide Average Mass—4813.4 Observed Mass—1205 (M+4H)⁺⁴

### Example 10: Preparation of Tirzepatide

Fmoc-deblocking of 15 g/5 mm Fmoc-(30-39)-Resin (Example 7) was done using piperidine/DMF. The mixture was stirred for 30 minute and then washed with a solvent to get to pH neutral. Checked in-process test for confirmation of Fmoc-removal from the resin. This is H-Gly³⁰-Pro-Ser(tBu)-Ser(tBlu)-Gly-Ala-Pro-Pro-Pro-Ser³⁹-Resin (H-30-39-RESIN),

Next amino acid/Fragment was coupled as per sequence using solid phase peptide synthesis including deblocking, coupling, and capping. Details of equivalent of protected amino acid/fragment, reagent and solvent were as below:

| S. No. | Amino Acid | Condition |
|---|---|---|
| 1. | Fmoc-Gly-OH | Deblocking |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 3 Hrs RT |
| 2. | Fmoc-Ala-OH | Deblocking |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 3 Hrs RT |
| 3. | Fmoc-Ile-OH | Deblocking |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 3 Hrs RT |
| 4. | Fmoc-Leu-OH | Deblocking |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 3 Hrs RT |
| 5. | Fmoc-Trp-OH | Deblocking |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 3 Hrs RT |
| 6. | Fmoc-Gln(Trt)-OH | Deblocking |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 3 Hrs RT |
| 7. | Fmoc-Val-OH | Deblocking |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 3 Hrs RT |
| 8. | Fmoc-Phe-OH | Deblocking |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 3 Hrs RT |
| 9. | Fmoc-Lys(Side chain)-Ala- | Coupling- |
| | OH | Solvent-DMF |
| | | 1.6 eq AA/1.6 eq DIPC/1.6 eq HOBt, 24 Hrs RT |
| 10. | Fmoc-Gln(Trt)-OH | Deblocking |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 3 Hrs RT |
| 11. | Fmoc-Ala-OH | Deblocking |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 3 Hrs RT |
| 12. | Fmoc-Ile-OH | Deblocking |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 6 Hrs RT |
| 13. | Fmoc-Lys(Boc)-OH | Deblocking |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 3 Hrs RT |
| 14. | Fmoc-Asp(OtBu)-OH | Deblocking |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 3 Hrs RT |
| 15. | Fmoc-Asp(OtBu)9- | Deblocking |
| | Tyr(tBu)-Ser(tBu)-Ile- | 50% Piperidine/DMF 30 min RT |
| | Aib-Leu-OH | Check pH |
| | | Coupling- |
| | | Solvent-Tetrahydrofuran/DMF |
| | | 2.0 eq AA/2 eq HOBt/2.0 eq DIPC, 4 Hrs RT |
| 16. | Fmoc-Thr(tBu)5-Phe- | Deblocking |
| | Thr(tBu)-Ser(tBu)8-OH | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 4 Hrs RT |
| 17. | Boc-Tyr(tBu)1-Aib- | Deblocking- |
| | Glu(OtBu)-Gly-OH | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 4 Hrs RT |

On completion of solid phase synthesis the resin was removed from the assembly and dried to get Boc-Tyr(tBlu)1-Aib-Glu(OtBu)-Gly-Thr(tBlu)⁵-Phe-Thr(tBu)-Ser(tBu)-Asp(OtBu)-Tyr(tBlu)¹⁰-Ser(tBu)-Ile-Aib-Leu-Asp(OtBu)¹⁵-Lys(Boc)-Ile-Ala-Gln(Trt)-Lys²⁰(Side chain)-Ala-Phe-Val-Gln(Trt)-Trp²⁵-Leu-Ile-Ala-Gly-Gly30-Pro-Ser(tBu)-Ser(tBu)-Gly-Ala35-Pro-Pro-Pro-Ser(tBlu)³⁹-Rink Resin-21 g (Boc-(01-39)-Rink Resin).

20 g Boc-(01-39)-Rink Resin was washed with dichloromethane and suspended in 200 mL TFA/ethanedithiol (95:5) for 1 hour at temperature of 20 to 30° C. The reaction mixture was suction dried and the filtrate was collected. The resin was further washed with 2×25 mL TFA and mixed with the filtrate.

The filtrate was distilled out on a rotavapor at 20-30° C. to get a sticky syrup. The sticky syrup was triturated with 100 mL diethylether and further washed with diethyl ether to get a crude solid −10 g.

### Purification of Tirzepatide

The tirzepatide peptide crude 8 g obtained above was dissolved in a buffer of pH 8.5 and acetonitrile. The tirzepatide crude peptide solution was purified on octadecyl bonded silica gel with a gradient of a phosphate buffer of pH 8 and acetonitrile. The tirzepatide enriched fraction was again purified with TFA in water and acetonitrile, and the impure pool was further purified with a phosphate buffer of pH 2.5 and acetonitrile. The pure fraction of tirzepatide was desalted as a sodium salt, and the acetonitrile was removed from eluent by distillation over a rotavapor. The concentrated solution was taken for freeze drying to obtain 900 mg tirzepatide with 99.47% HPLC purity.

Tirzepatide Average Mass—4813.4 Observed Mass—1205 (M+4H)⁺⁴

### Example 11: Preparation of Tirzepatide

Fmoc-deblocking of 15 g/5 mm Fmoc-(30-39)-Resin (Example 7) was done using piperidine/DMF. The mixture was stirred for 30 minutes and the washed with a solvent to get to pH neutral. Checked in-process test for confirmation of Fmoc-removal from the resin. This is H-Gly³⁰-Pro-Ser(tBu)-Ser(tBu)-Gly-Ala-Pro-Pro-Pro-Ser³⁹-Resin (H-30-39-RESIN).

Next amino acid/Fragment was coupled as per sequence using solid phase peptide synthesis including deblocking, coupling, and capping. Details of equivalent of protected amino acid/fragment, reagent and solvent were as below:

| S. No. | Amino Acid | Condition |
|---|---|---|
| 1. | Fmoc-Gly-OH | Deblocking |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 3 Hrs RT |
| 2. | Fmoc-Ala-OH | Deblocking |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 3 Hrs RT |
| 3 | Fmoc-Ile-OH | Deblocking |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 3 Hrs RT |
| 4. | Fmoc-Leu-OH | Deblocking |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 3 Hrs RT |
| 5. | Fmoc-Trp-OH | Deblocking |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 3 Hrs RT |
| 6. | Fmoc-Gln(Trt)-OH | Deblocking |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 3 Hrs RT |
| 7. | Fmoc-Val-OH | Deblocking |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 3 Hrs RT |
| 8. | Fmoc-Phe-OH | Deblocking |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 3 Hrs RT |
| 9. | Fmoc-Lys(Side chain)-Ala- | Coupling- |
| | OH | Solvent-DMF |
| | | 1.6 eq AA/1.6 eq DIPC/1.6 eq HOBt, 24 Hrs RT |
| 10. | Fmoc-Gln(Trt)-OH | Deblocking |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 3 Hrs RT |
| 11. | Fmoc-Ala-OH | Deblocking |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 3 Hrs RT |
| 12. | Fmoc-Ile-OH | Deblocking |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 6 Hrs RT |
| 13. | Fmoc-Lys(Boc)-OH | Deblocking |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 3 Hrs RT |
| 14. | Fmoc-Asp(OtBu)-OH | Deblocking |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 3 Hrs RT |
| 15. | Fmoc-Asp(OtBu)9- | Deblocking |
| | Tyr(tBu)-Ser(tBu)-Ile- | 50% Piperidine/DMF 30 min RT |
| | Aib-Leu-OH | Check pH |
| | | Coupling- |
| | | Solvent-Tetrahydrofuran/DMF |
| | | 2.0 eq AA/2 eq HOBt/2.0 eq DIPC, 4 Hrs RT |
| 16. | Boc-Tyr(tBu)1-Aib- | Deblocking- |
| | Glu(OtBu)-Gly-Thr(tBu)5- | 50% Piperidine/DMF 30 min RT |
| | Phe-Thr(tBu)-Ser(tBu)8- | Check pH |
| | OH | Coupling- |
| | | Solvent-Tetrahydrofuran/DMF |
| | | 2.0 eq AA/2 eq HOBt/2.0 eq DIPC, 8 Hrs RT |

On completion of solid phase synthesis, the resin was removed from the assembly and dried to get Boc-Tyr(tBu)¹-Aib-Glu(OtBu)-Gly-Thr(tBu)⁵-Phe-Thr(tBu)-Ser(tBu)-Asp(OtBu)-Tyr(tBu)¹⁰-Ser(tBu)-Ile-Aib-Leu-Asp(OtBu)¹⁵-Lys(Boc)-Ile-Ala-Gln(Trt)-Lys²⁰(Side chain)-Ala-Phe-Val-Gln(Trt)-Trp²⁵-Leu-Ile-Ala-Gly-Gly30-Pro-Ser(tBu)-Ser(tBu)-Gly-Ala³⁵-Pro-Pro-Pro-Ser(tBu)³⁹-Rink Resin-18 g (Boc-(01-39)-Rink Resin).

15 g Boc-(01-39)-Rink Resin was washed with dichloromethane and suspended in 200 mL TFA/ethanedithiol (95:5) for 1 hour at temperature a of 20 to 30° C. The reaction mixture was suction dried and the filtrate was collect. The resin was further washed with 2×25 mL TFA and mixed with the filtrate.

Distilled out the filtrate on a rotavapor at 20-30° C. to get a sticky syrup. The sticky syrup was triturated with 100 mL diethylether and then further washed with diethyl ether to get a crude solid −8 g.

### Purification of Tirzepatide

The tirzepatide peptide crude 8 g obtained above was dissolved in a buffer of pH 8.5 and acetonitrile. The tirzepatide crude peptide solution was purified on octadecyl bonded silica gel with a gradient of phosphate buffer of pH 8 and acetonitrile. The tirzepatide enriched fraction was again purified with TFA in water and acetonitrile. The impure pool was further purified with a phosphate buffer of pH 2.5 and acetonitrile. The pure fraction of tirzepatide was desalted as a sodium salt, and the acetonitrile was removed from the eluent by distillation over a rotavapor. The concentrated solution was taken for freeze drying to obtain 800 mg tirzepatide with 99.13% HPLC purity.

Tirzepatide Average Mass—4813.4 Observed Mass—1205 (M+4H)⁺⁴

### Example 12: Preparation of Tirzepatide

Fmoc-deblocking of 15 g/mm Fmoc-(30-39)-Resin (Example 7) was done using piperidine/DMF. The mixture was stirred for 30 minute and washed with a solvent to get to pH neutral. Checked in-process test for confirmation of Fmoc-removal from the resin. This is H-30-39-RESIN.

Next amino acid/Fragment was coupled as per sequence using solid phase peptide synthesis including deblocking, coupling, and capping. Details of equivalent of protected amino acid/fragment, reagent and solvent were as below.

| S. No. | Amino Acid | Condition |
|---|---|---|
| 1. | Fmoc-Gly-OH | Deblocking |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 3 Hrs RT |
| 2. | Fmoc-Ala-OH | Deblocking |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 3 Hrs RT |
| 3. | Fmoc-Ile-OH | Deblocking |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 3 Hrs RT |
| 4. | Fmoc-Leu-OH | Deblocking |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 3 Hrs RT |
| 5. | Fmoc-Trp-OH | Deblocking |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 3 Hrs RT |
| 6. | Fmoc-Gln(Trt)-OH | Deblocking |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 3 Hrs RT |
| 7. | Fmoc-Val-OH | Deblocking |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 3 Hrs RT |
| 8. | Fmoc-Phe-OH | Deblocking |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 3 Hrs RT |
| 9. | Fmoc-Ala-OH | Deblocking |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 3 Hrs RT |
| 10. | Fmoc-Lys(Side chain)-OH | Coupling- |
| | | Solvent-DMF |
| | | 1.6 eq AA/1.6 eq DIPC/1.6 eq HOBt, 24 Hrs RT |
| 11. | Fmoc-Gln(Trt)-OH | Deblocking |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 3 Hrs RT |
| 12. | Fmoc-Ala-OH | Deblocking |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 3 Hrs RT |
| 13. | Fmoc-Ile-OH | Deblocking |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 6 Hrs RT |
| 14. | Fmoc-Lys(Boc)-OH | Deblocking |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 3 Hrs RT |
| 15. | Fmoc-Asp(OtBu)-OH | Deblocking |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 3 Hrs RT |
| 16. | Fmoc-Leu-OH | Deblocking- |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 3 Hrs RT |
| 17. | Fmoc-Aib-OH | Deblocking- |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 3 Hrs RT |
| 18. | Fmoc-Ile-OH | Deblocking- |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 12 Hrs RT |
| 19. | Fmoc-Ser(tBu)-OH | Deblocking- |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 3 Hrs RT |
| 20. | Fmoc-Tyr(tBu)-OH | Deblocking- |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 3 Hrs RT |
| 21. | Fmoc-Asp(OtBu)-OH | Deblocking- |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 3 Hrs RT |
| 22. | Fmoc-Ser(tBu)-OH | Deblocking- |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 3 Hrs RT |
| 23. | Fmoc-Thr(tBu)-OH | Deblocking- |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 3 Hrs RT |
| 24. | Fmoc-Phe-OH | Deblocking- |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 3 Hrs RT |
| 25. | Fmoc-Thr(tBu)-OH | Deblocking- |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 3 Hrs RT |
| 26. | Fmoc-Gly-OH | Deblocking- |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 3 Hrs RT |
| 27. | Fmoc-Glu(OtBu)-OH | Deblocking- |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 3 Hrs RT |
| 28. | Fmoc-Aib-OH | Deblocking |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 4 Hrs RT |
| 29. | Boc-Tyr(tBu)-OH | Deblocking- |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 6 Hrs RT |

On completion of solid phase synthesis, the resin was removed from the assembly and dried to get Boc-Tyr(tBu)¹-Aib-Glu(OtBu)-Gly-Thr(tBu)⁵-Phe-Thr(tBu)-Ser(tBu)-Asp(OtBu)-Tyr(tBu)¹⁰-Ser(tBu)-Ile-Aib-Leu-Asp(OtBu)¹⁵-Lys(Boc)-Ile-Ala-Gln(Trt)-Lys²⁰(Side chain)-Ala-Phe-Val-Gln(Trt)-Trp²⁵-Leu-Ile-Ala-Gly-Gly30-Pro-Ser(tBu)-Ser(tBu)-Gly-Ala³⁵-Pro-Pro-Pro-Ser(tBu)³⁹-Rink Resin-30 g (Boc-(01-39)-Rink Resin).

20 g Boc-(01-39)-Rink Resin was washed with dichloromethane and suspended in 200 mL TFA/ethanedithiol (95:5) for 1 hour at temperature of 20 to 30° C. The reaction mixture was suction dried and the filtrate was collected. The resin was further washed with 2×25 mL TFA and mixed with the filtrate.

Distilled out filtrate on a rotavapor at 20-30° C. to get a sticky syrup. The sticky syrup was triturated with 100 mL diethylether and further washed with diethyl ether to get a crude solid-10 g.

### Purification of Tirzepatide

The tirzepatide peptide crude 10 g obtained above was dissolved in a buffer of pH 8.5 and acetonitrile. The tirzepatide crude peptide solution was purified on octadecyl bonded silica gel with a gradient of a phosphate buffer of pH 8 and acetonitrile. The tirzepatide enriched fraction was again purified with TFA in water and acetonitrile, and the impure pool was further purified with a phosphate buffer of pH 2.5 and acetonitrile. The pure fraction of tirzepatide was desalted as an acetate salt, and the acetonitrile was removed from the eluent by distillation over a rotavapor. The concentrated solution was taken for freeze drying to obtain 1.5 g tirzepatide with 99.07% HPLC purity.

Tirzepatide Average Mass—4813.4 Observed Mass—1205 (M+4H)⁺⁴

### Example 13: Preparation of Tirzepatide

Fmoc-deblocking of 15 g/5 mm Fmoc-(30-39)-Resin (Example 7) was done using piperidine/DMF. The mixture was stirred for 30 minute and then washed with solvent to get to pH neutral. Checked in-process test for confirmation of Fmoc-removal from the resin. This is H-Gly30-Pro-Ser(tBu)-Ser(tBu)-Gly-Ala-Pro-Pro-Pro-Ser39-Resin (H-30-39-RESIN).

Next amino acid/Fragment was coupled as per sequence using solid phase peptide synthesis including deblocking, coupling, and capping. Details of equivalent of protected amino acid/fragment, reagent and solvent were as below:

| S. No. | Amino Acid | Condition |
|---|---|---|
| 1. | Fmoc-Ile12-Aib-Leu- | Coupling- |
| | Asp(OtBu)-Lys(Boc)- | Solvent-DMF |
| | Ile-Ala18-Gln(Trt)- | 1.6 eq AA/1.6 eq DIPC/1.6 eq |
| | Lys20(Side chain)- | HOBt, 24 Hrs RT |
| | Ala-Phe-Val- | |
| | Gln(Trt)-Trp25- | |
| | Leu-Ile-Ala-Gly29- | |
| | OH | |
| 2. | Boc-Tyr(tBu)1-Aib- | Deblocking- |
| | Glu(OtBu)-Gly- | 50% Piperidine/DMF 30 min RT |
| | Thr(tBu)5-Phe- | Check pH |
| | Thr(tBu)-Ser(tBu)8- | Coupling- |
| | Asp(OtBu)-Tyr(tBu)- | Solvent-Tetrahydrofuran/DMF |
| | Ser(tBu)-OH | 1.6 eq AA/1.6 eq HOBt/1.6 eq |
| | | DIPC, 24 Hrs RT |

On completion of solid phase synthesis, the resin was removed from the assembly and dried to get Boc-Tyr(tBu)¹-Aib-Glu(OtBu)-Gly-Thr(tBu)⁵-Phe-Thr(tBu)-Ser(tBu)-Asp(OtBu)-Tyr(tBu)¹⁰-Ser(tBu)-Ile-Aib-Leu-Asp(OtBu)¹⁵-Lys(Boc)-Ile-Ala-Gln(Trt)-Lys²⁰(Side chain)-Ala-Phe-Val-Gln(Trt)-Trp²⁵-Leu-Ile-Ala-Gly-Gly³⁰-Pro-Ser(tBu)-Ser(tBu)-Gly-Ala³⁵-Pro-Pro-Pro-Ser(tBu)³⁹-Rink Resin-21 g (Boc-(01-39)-Rink Resin).

20 g Boc-(01-39)-Rink Resin was washed with dichloromethane and suspended in 200 mL TFA/ethanedithiol (95:5) for 1 hour at a temperature of 20 to 30° C. The reaction mixture was suction dried and the filtrate was collected. The resin was further washed with 2×25 mL TFA and mixed with the filtrate.

Distilled out the filtrate on a rotavapor at 20-30° C. to get a sticky syrup. The sticky syrup was triturated with 100 mL diethylether and further washed with diethyl ether to get a crude solid −10 g.

### Purification of Tirzepatide

The tirzepatide peptide crude 10 g obtained above was dissolved in a buffer of pH 8.5 and acetonitrile. The tirzepatide crude peptide solution was purified on octadecyl bonded silica gel with a gradient of phosphate buffer of pH 8 and acetonitrile. The tirzepatide enriched fraction was again purified with TFA in water and acetonitrile, and the impure pool was further purified with a phosphate buffer of pH 2.5 and acetonitrile. The pure fraction of tirzepatide was desalted as a sodium salt, and acetonitrile was removed from the eluent by distillation over a rotavapor. The concentrated solution was taken for freeze drying to obtain 800 mg Tirzepatide with 99.46% HPLC purity.

Tirzepatide Average Mass—4813.4 Observed Mass—1205 (M+4H)⁺⁴

### Example 14: Preparation of Tirzepatide

Fmoc-deblocking of 15 g/5 mm Fmoc-(30-39)-Resin (Example 7) was done using piperidine/DMF. The mixture was stirred for 30 minute and washed with a solvent to get to pH neutral. Checked in-process test for confirmation of Fmoc-removal from the resin. This is H-30-39-RESIN.

Next amino acid/Fragment was coupled as per sequence using solid phase peptide synthesis including deblocking, coupling, and capping. Details of equivalent of protected amino acid/fragment, reagent and solvent were as below:

| S. No. | Amino Acid | Condition |
|---|---|---|
| 1. | Fmoc-Lys(Boc)16-Ile- | Coupling- |
| | Ala18-Gln(Trt)- | Solvent-DMF |
| | Lys20(Side chain)-Ala-Phe- | 1.6 eq AA/1.6 eq DIPC/1.6 eq HOBt, 24 Hrs RT |
| | Val-Gln(Trt)-Trp25- | |
| | Leu-Ile-Ala-Gly29-OH | |
| 2. | Fmoc-Asp(OtBu)-OH | Deblocking- |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 3 Hrs RT |
| 3. | Fmoc-Leu-OH | Deblocking- |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 3 Hrs RT |
| 4. | Fmoc-Aib-OH | Deblocking- |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 3 Hrs RT |
| 5. | Fmoc-Ile-OH | Deblocking- |
| | | 50% Piperidine/DMF 30 min RT |
| | | Check pH |
| | | Coupling- |
| | | Solvent-Tetrahydrofuran/DMF |
| | | 2.0 eq AA/2 eq HOBt/2.0 eq DIPC, 24 Hrs RT |
| 6. | Boc-Tyr(tBu)1-Aib- | Deblocking- |
| | Glu(OtBu)-Gly-Thr(tBu)5- | 50% Piperidine/DMF 30 min RT |
| | Phe-Thr(tBu)-Ser(tBu)8- | Check pH |
| | Asp(OtBu)-Tyr(tBu)- | Coupling- |
| | Ser(tBu)-OH | Solvent-Tetrahydrofuran/DMF |
| | | 1.6 eq AA/1.6 eq HOBt/1.6 eq DIPC, 24 Hrs RT |

On completion of solid phase synthesis, the resin was removed from the assembly and dried to get Boc-Tyr(tBlu)¹-Aib-Glu(OtBu)-Gly-Thr(tBlu)⁵-Phe-Thr(tBu)-Ser(tBlu)-Asp(OtBu)-Tyr(tBlu)¹⁰-Ser(tBu)-Ile-Aib-Leu-Asp(OtBu)¹⁵-Lys(Boc)-Ile-Ala-Gln(Trt)-Lys²⁰(Side chain)-Ala-Phe-Val-Gln(Trt)-Trp²⁵-Leu-Ile-Ala-Gly-Gly30-Pro-Ser(tBu)-Ser(tBu)-Gly-Ala³⁵-Pro-Pro-Pro-Ser(tBu)³⁹-Rink Resin-22 g (Boc-(01-39)-Rink Resin).

20 g Boc-(01-39)-Rink Resin was washed with dichloromethane and suspended in 200 mL TFA/ethanedithiol (95:5) for 1 hour at a temperature of 20 to 30° C. The reaction mixture was suction dried and the filtrate was collect filtrate. The resin was further washed with 2×25 mL TFA and mixed with the filtrate.

Distilled out the filtrate on a rotavapor at 20-30° C. to get a sticky syrup. The sticky syrup was triturated with 100 mL diethylether and further washed with diethyl ether to get a crude solid −10 g.

### Purification of Tirzepatide

The tirzepatide peptide crude 10 g obtained above was dissolved in a buffer of pH 8.5 and acetonitrile. The tirzepatide crude peptide solution was purified on octadecyl bonded silica gel with a gradient of phosphate buffer of pH 8 and acetonitrile. The tirzepatide enriched fraction was again purified with TFA in water and acetonitrile, and the impure pool was further purified with a phosphate buffer of pH 2.5 and acetonitrile. The pure fraction of tirzepatide was desalted as an acetate salt, and the acetonitrile was removed from the eluent by distillation over a rotavapor. The concentrated solution was taken for freeze drying to obtain 900 mg Tirzepatide with 99.74% HPLC purity.

Tirzepatide Average Mass—4813.4 Observed Mass—1205 (M+4H)⁺⁴

### Example 15: Preparation of Sodium salt of Tirzepatide

Loaded 0.5 g Tirzepatide solution on a PREP column. The column was washed with 1 L of a 0.001N sodium hydroxide solution. Tirzepatide was eluted from the column with a water and acetonitrile gradient. The eluent was collected and the acetonitrile was distilled out on a rotavapour. The remaining eluent was freeze dried for 72 hrs to obtain a sodium salt of tirzepatide.

The sodium content in the product was determined using ion chromatography by liquid chromatogram.

### Example 16: Preparation of Potassium Salt of Tirzepatide

Loaded about 0.5 g of a tirzepatide solution on PREP column. The column was washed with 1.5 Liters of a 0.001 N potassium hydroxide solution, and tirzepatide was eluted from the column with a water and acetonitrile gradient. The eluent was collect and the acetonitrile was distilled out on a rotavapour. The remaining eluent was freeze dried for 72 hrs to obtain a potassium salt of tirzepatide.

The potassium content in the product was determined using ion chromatography by liquid chromatogram.

### Example 17: Preparation of Ammonium Salt of Tirzepatide

Loaded about 0.5 g Tirzepatide solution on a PREP column. The column was washed with 2 L of a 3% ammonium acetate solution, follow by 2 L of a water wash. The tirzepatide was eluted from the column with a water and acetonitrile gradient. The eluent was collected and the acetonitrile was distilled out on a rotavapour. The remaining eluent was freeze dried for 72 hrs to obtain an ammonium salt of tirzepatide.

The ammonium content in the product was determined using ion chromatography by liquid chromatogram.

### Example 18: Preparation of Acetate Salt of Tirzepatide

Loaded about 1 g tirzepatide solution on a PREP column. The column was washed with 2 L of 1% acetic acid in water. The tirzepatide was eluted from the column with 1% acetic acid in water and acetonitrile gradient. The eluent was collected and the acetonitrile was distilled out on a rotavapour. The remaining eluent was freezed dried for 72 hrs to obtain an acetate salt of tirzepatide.

The acetate content in the product was determined using HPLC.

We claim:

1. A process for the preparation of tirzepatide or a pharmaceutically acceptable salt thereof, comprising use of at least one polypeptide or a pharmaceutically acceptable salt thereof selected from the group consisting of SEQ ID NO. 1, SEQ ID NO. 2, SEQ ID NO. 3, SEQ ID NO. 4, SEQ ID NO. 5, SEQ ID NO. 6, SEQ ID NO. 7, SEQ ID NO. 8 or SEQ ID NO. 10.

2. The process as claimed in claim 1, wherein the process further comprises use of a polypeptide or pharmaceutically acceptable salt of SEQ ID NO. 9.

3. The process as claimed in claim 1, wherein the process comprises the use of polypeptides of SEQ ID NO. 1, SEQ ID NO. 2, SEQ ID NO. 3 and SEQ ID NO. 9.

4. The process as claimed in claim 1, wherein the process comprises the use of polypeptides of SEQ ID NO. 1, SEQ ID NO. 2, SEQ ID NO. 4 and SEQ ID NO. 9.

5. The process as claimed in claim 1, wherein the process comprises use of polypeptides of SEQ ID NO. 5, SEQ ID NO. 2 and SEQ ID NO. 4.

6. The process as claimed in claim 1, wherein the process comprises use of polypeptides of SEQ ID NO. 6 and SEQ ID NO. 7.

7. The process as claimed in claim 1, wherein the process comprises use of polypeptides of SEQ ID NO. 6 and SEQ ID NO. 10.

8. The process as claimed in claim 1, wherein the process comprises use of polypeptides of SEQ ID NO. 6 and SEQ ID NO. 8.

9. Tirzepatide or a pharmaceutically acceptable salt thereof prepared by a process comprising use of at least one polypeptide or a pharmaceutically acceptable salt thereof selected from the group consisting of SEQ ID NO. 1, SEQ ID NO. 2, SEQ ID NO. 3, SEQ ID NO. 4, SEQ ID NO. 5, SEQ ID NO. 6, SEQ ID NO. 7, SEQ ID NO. 8 or SEQ ID NO. 10.

10. Tirzepatide or a pharmaceutically acceptable salt thereof as claimed in claim 9, wherein the tirzepatide or a pharmaceutically acceptable salt comprises not more than 0.5% of a single major impurity.

11. Tirzepatide or a pharmaceutically acceptable salt as claimed in claim 10, wherein the major impurity is a compound of SEQ ID NO. 11, SEQ ID NO. 12, SEQ ID NO. 13, SEQ ID NO. 15, SEQ ID NO. 17, SEQ ID NO. 18, SEQ ID NO. 19, SEQ ID NO. 20, SEQ ID NO. 21, SEQ ID NO. 22, SEQ ID NO. 23 or SEQ ID NO. 24 or a pharmaceutically acceptable salt thereof.

12. Tirzepatide or a pharmaceutically acceptable salt comprising less than 1% of a compound of SEQ ID NO. 22.

13. Tirzepatide or a pharmaceutically acceptable salt comprising less than 0.5% of a compound of SEQ ID NO. 22.

14. A compound of SEQ ID NO. 1 or a pharmaceutically acceptable salt thereof.

15. A compound of SEQ ID NO. 2 or a pharmaceutically acceptable salt thereof.

16. A compound of SEQ ID NO. 3 or a pharmaceutically acceptable salt thereof.

17. A compound of SEQ ID NO. 4 or a pharmaceutically acceptable salt thereof.

18. A compound of SEQ ID NO. 5 or a pharmaceutically acceptable salt thereof.

19. A compound of SEQ ID NO. 6 or a pharmaceutically acceptable salt thereof.

20. A compound of SEQ ID NO. 7 or a pharmaceutically acceptable salt thereof.

21. A compound of SEQ ID NO. 8 or a pharmaceutically acceptable salt thereof.

22. A compound of SEQ ID NO. 10 or a pharmaceutically acceptable salt thereof.

23. A compound of SEQ ID NO. 11 or a pharmaceutically acceptable salt thereof.

24. A compound of SEQ ID NO. 12 or a pharmaceutically acceptable salt thereof.

25. A compound of SEQ ID NO. 13 or a pharmaceutically acceptable salt thereof.

26. A compound of SEQ ID NO. 15 or a pharmaceutically acceptable salt thereof.

27. A compound of SEQ ID NO. 17 or a pharmaceutically acceptable salt thereof.

28. A compound of SEQ ID NO. 18 or a pharmaceutically acceptable salt thereof.

29. A compound of SEQ ID NO. 19 or a pharmaceutically acceptable salt thereof.

30. A compound of SEQ ID NO. 20 or a pharmaceutically acceptable salt thereof.

31. A compound of SEQ ID NO. 21 or a pharmaceutically acceptable salt thereof.

32. A compound of SEQ ID NO. 22 or a pharmaceutically acceptable salt thereof.

33. A compound of SEQ ID NO. 23 or a pharmaceutically acceptable salt thereof.

34. A compound of SEQ ID NO. 24 or a pharmaceutically acceptable salt thereof.

35. A compound of SEQ ID NO. 1, SEQ ID NO. 2, SEQ ID NO. 3, SEQ ID NO. 4, SEQ ID NO. 5, SEQ ID NO. 6, SEQ ID NO. 7, SEQ ID NO. 8, SEQ ID NO. 10 or pharmaceutically acceptable salt thereof for use in the synthesis of tirzepatide or a pharmaceutically acceptable salt thereof.

36. A compound that is a sodium salt of tirzepatide.

37. The sodium salt of tirzepatide as claimed in claim 36, wherein the sodium content is from 1.5% to 2.5%.

38. A compound that is a potassium salt of tirzepatide.

39. A compound that is an ammonium salt of tirzepatide.

40. A compound that is an acetate salt of tirzepatide.

41. Tirzepatide or a pharmaceutically acceptable salt thereof that is substantially free from impurities selected from the group consisting of SEQ ID NO. 11, SEQ ID NO. 12, SEQ ID NO. 13, SEQ ID NO. 15, SEQ ID NO. 17, SEQ ID NO. 18, SEQ ID NO. 19, SEQ ID NO. 20, SEQ ID NO. 21, SEQ ID NO. 22, SEQ ID NO. 23 or SEQ ID NO. 24.

42. Tirzepatide or a pharmaceutically acceptable salt thereof that is substantially free of an impurity of SEQ ID NO. 22.

43. A pharmaceutical composition consisting essentially of Tirzepatide or a pharmaceutically acceptable salt thereof as claimed in claim 41 or 42.

44. The pharmaceutically acceptable salt according to any of the proceeding claims wherein the salt is selected from a sodium, potassium, ammonium and acetate salt of tirzepatide.
